(19) United States
(12) Patent Application Publication
(10) Pub. No.: US 2025/0279203 A1
(43) Pub. Date: Sep. 4, 2025

(54) Digital Care System Based on Sensing and Monitoring Patient's Mobility

(71) Applicant: Celloscope Ltd., Tel Aviv (IL)

(72) Inventors: Yuval NAVEH, Haifa (IL); Tamir ALOUSH, Kiryat Ono (IL); Levi TEITZ, Tel Aviv (IL); Shahar DAVIDSON, Rehovot (IL); Tomer SHUSSMAN, Kfar Saba (IL); Hila GLICK, Ramat Hasharon (IL); Itamar BEN DAVID, Giv'atayim (IL); Guy KEREM, Kiryat Ono (IL)

(21) Appl. No.: 19/062,758

(22) Filed: Feb. 25, 2025

Related U.S. Application Data
(63) Continuation-in-part of application No. 18/939,288, filed on Nov. 6, 2024.
(63) Continuation-in-part of application No. 18/975,890, filed on Dec. 10, 2024.
(63) Continuation-in-part of application No. 19/049,400, filed on Feb. 10, 2025.
(63) Continuation-in-part of application No. 19/051,712, filed on Feb. 12, 2025.
(63) Continuation-in-part of application No. 19/056,378, filed on Feb. 18, 2025.
(60) Provisional application No. 63/596,479, filed on Nov. 6, 2023.
(60) Provisional application No. 63/612,587, filed on Dec. 20, 2023.
(60) Provisional application No. 63/557,740, filed on Feb. 26, 2024.
(60) Provisional application No. 63/557,747, filed on Feb. 26, 2024.
(60) Provisional application No. 63/557,753, filed on Feb. 26, 2024.
(60) Provisional application No. 63/557,757, filed on Feb. 26, 2024.
(60) Provisional application No. 63/557,762, filed on Feb. 26, 2024.

(51) Int. Cl. G16H 50/20; A61B 5/00; G16H 10/20; G16H 40/67; G16H 50/30; G16H 80/00

(52) U.S. Cl. CPC G16H 50/20; A61B 5/7267; A61B 5/7475; G16H 10/20; G16H 40/67; G16H 50/30; G16H 80/00; A61B 2562/0219

(57) ABSTRACT

Computerized healthcare method providing healthcare remotely to end-users, the method comprising providing at least one instance of an interface uploading IMU data recorded by a healthcare providing organization O's end users' communication devices' accelerometers; and/or providing at least one instance (e.g. one per organization in an eco-system of organizations which may refer patients one to another) of a hardware processor which maps indications derived from the IMU data to care actions. The care actions typically comprise allocation/s of organizational resource/s from among a typically finite set of available organizational resources which the organization allocates to its end-users or patients or members, to reliably and efficiently allocate available organizational resources to at least one organization's patients.

### CROSS-REFERENCE TO RELATED APPLICATIONS

The present application is a continuation-in-part of U.S. application Ser. No. 18/939,288, filed Nov. 6, 2024, and of U.S. application Ser. No. 18/975,890, filed Dec. 10, 2024, and of U.S. application Ser. No. 19/049,400, filed Feb. 10, 2025, and of U.S. application Ser. No. 19/051,712 entitled “System, Method, and Computer Program Product for Tracking Cognitive Status Based On Repeated Mobility Assessments” filed Feb. 12, 2025, and of U.S. patent application Ser. No. 19/056,378 filed Feb. 18, 2025 and of U.S. patent application Ser. No. 19/061,254 filed Feb. 24, 2025, the entire contents of each of which being hereby fully incorporated herein by reference. The present application further claims benefit, directly or indirectly, of the following provisional applications, the entire contents of each of which being fully incorporated herein by reference: Application No. 63/557,740, filed Feb. 26, 2024 and entitled “Mobility Assessment in Visually Impaired Individuals”; Application No. 63/557,747, filed Feb. 26, 2024; Application No. 63/557,753, filed Feb. 26, 2024; Application No. 63/557,757, filed Feb. 26, 2024, Application No. 63/557,762, filed Feb. 26, 2024; Application No. 63/596,479, filed Nov. 6, 2023; and Application No. 63/612,587, filed Dec. 20, 2023.

### FIELD OF THIS DISCLOSURE

The present invention relates generally to computerized analysis of motion, and more particularly to computerized analysis of human motion which receives sensor outputs borne by a human, typically in real time.

### BACKGROUND FOR THIS DISCLOSURE

OneStep is an FDA-listed medical app, downloadable from GooglePlay, that uses smartphone motion sensors to provide immediate, clinically-validated feedback on gait inter alia.

The disclosures of all publications and patent documents mentioned above and elsewhere in the specification, and of the publications and patent documents cited therein directly or indirectly, are hereby incorporated herein by reference in their entirety. If the incorporated material is inconsistent with the express disclosure herein, the interpretation is that the express disclosure herein describes certain embodiments, whereas the incorporated material describes other embodiments. Definition/s within the incorporated material may be regarded as one possible definition for the term/s in question. www.medicalalertbuyersguide.org/best-medical-alert-smartwatch-system/, reviews “medical alert watches for seniors”. Some watches have “automatic fall detection”.

### SUMMARY OF CERTAIN EMBODIMENTS

Certain embodiments of the present invention seek to provide circuitry typically comprising at least one processor in communication with at least one memory, with instructions stored in such memory executed by the processor to provide functionalities which are described herein in detail. Any functionality described herein may be firmware-implemented or processor-implemented, as appropriate.

Certain embodiments provide a system which enables organizational end-users (each organizational end-user itself has end-users/patients and may for example comprise an HMO aka health maintenance organization) to continuously screen for new medical issues newly afflicting the organization's patients and, via organizational rules, to assign interventions accordingly. For example, the organization's personnel may have been assigned to see 50 users on a certain day. However, due to a new medical issue that has materialized vis-a-vis user Julia (has been derived from Julia's phone's IMU data), personnel may need to be assigned to seeing or at least contacting Julia, and the lowest priority among the 50 users may be postponed to another day.

Typically, prioritization scores may be assigned to users to reflect how urgent a certain intervention, e.g. an in-clinic visit, is for this user, relative to the urgency of interventions other users are waiting for, taking into account the utility of this intervention and/or the status of this user, factoring in all known medical issues afflicting this user, including issues identified during previous in-clinic sessions or elsewhere, and also including issues identified by processing the user's IMU (Inertial Measurement Unit) data.

Typically, each organization, aka organizational user, which is a user of the system herein, includes a hardware processor configured to repeatedly or periodically or occasionally or continuously screen for new medical issues, e.g. a new medical development which has befallen a patient, aka end-user Julia, and, via rules, may assign outcomes, e.g. a command, to allocate certain organizational resources, responsive to the new medical issue or development which has occurred to patient Julia.

Certain embodiments seek to provide a digital care system based on monitoring a patient's mobility. Each organization typically has its own end-users, each or many of which typically use, e.g. wear or bear, a mobile device with IMU, a mobile app, a caregiver dashboard, and a processor configured to receive data and communicate with the mobile app and the caregiver dashboard. The processor continuously assesses the mobile app's user functional status and enables an organization or caregiver to administer different outcomes, e.g. care actions based on at least one mobility measure. Typically, taking no action is one possible outcome of a new functional status or new development occurring to a given user, such as Julia.

At least the following embodiments may be provided:

Embodiment 1: A computerized healthcare system providing healthcare remotely to end-users, the system comprising at least one instance of: an interface uploading IMU data recorded by a healthcare-providing organization O's end users' communication devices' accelerometers; and/or a hardware processor which maps indications derived from the IMU data to care actions, where the care actions comprise at least one allocation of at least one organizational resource from among a set of available organizational resources which the organization allocates to its end-users or patients or members (wherein the resources may include care services performed within the organization e.g. assignment of organization hardware to end-users and/or may include referral to an external organization which performs care services which may include assignment of the external organization's hardware to end-users), thereby to reliably and efficiently allocate available organizational resources to at least one organization's patients.

At least one indication from among the indications derived from patient P's IMU data may comprise a putative new diagnosis for patient P such as a possible new stroke or possible new fall or possible new bout of influenza or possible new fracture.

Allocation to end-user/patient x, from a finite (at any given point in time) set of organizational resources that a given organization e.g. Hmo or hospital has, may include allocation of any of the following organizational resources, by way of non-limiting example: hardware or equipment or devices, clinical staff services, computer time, screening services, examinations and/or treatment performed within the organization or by referral to another organization, medications.

It is appreciated that alternatively, the processor may map raw accelerometer data including raw data as recorded by the accelerometers, rather than indications, to the resources.

Embodiment 2. The system of any preceding embodiment wherein the processor is configured to predict externally generated scores from raw data recorded by the accelerometers.

Embodiment 3. The system of any preceding embodiment wherein the processor comprises a biomarker or indication that estimates indexes directly from IMU data recorded by the accelerometers.

Embodiment 4. The system of any preceding embodiment wherein the processor comprises at least one classifier trained to predict the externally generated scores from the raw data recorded by the accelerometers.

Embodiment 5. The system of any preceding embodiment wherein the externally generated scores comprise at least one score generated by at least one human clinician and the classifier is trained using, for training data, scores generated by at least one human clinician for at least one individual, paired with raw data recorded from the at least one individual by the accelerometers.

Embodiment 6. The system of any preceding embodiment wherein the externally generated scores comprise at least one score derived from a patient questionnaire, and the classifier is trained using, for training data, scores derived from a patient questionnaire filled out by at least one individual, paired with raw data recorded from the at least one individual by the accelerometers.

Embodiment 7. The system of any preceding embodiment wherein the externally generated scores comprise scores indicative of a motor disease such as but not limited to, multiple sclerosis or Parkinson's or malfunction of lower extremities.

Embodiment 8. The system of any preceding embodiment wherein the externally generated scores comprise at least one of: UPDRS scores, PDQ scores, EDSS scores, PDDS scores, LEFS scores, EQ-5D scores, SF-36 or SF-12 scores.

It is appreciated that classifiers may be trained to (e.g. repeatedly or occasionally or continuously or periodically and/or upon being triggered) predict or estimate each of the above and, typically, store the estimates/predictions on the cloud to yield longitudinal data about each patient. This may be the case for, say, 7 classifiers to predict or estimate the above 7 scores, or for any other classifier/s trained to yield any indication described herein such as but not limited to predictions of clinical outcomes. Each classifier, generating any indication herein, may be operated periodically e.g. daily or hourly or when triggered by, say, new data becoming available or by a clinician being about to engage or have a session with a specific patient.

Embodiment 9. A system according to any preceding embodiment which includes plural instances of the interface (e.g. plural secure data channels) and/or of the processor, for plural healthcare providing organizations O1, . . . ON respectively, and wherein the organizational resources, which at least one organization Oj allocates to its end-users or patients or members, includes referrals to outside organizations, other than organization Oj, which typically perform care actions not performed within the organization Oj.

Embodiment 10. The system of any preceding embodiment wherein recording of IMU data is unobtrusive and/or frequent and/or continuous (e.g. does not require activation) and/or passive.

Embodiment 11. The system of any preceding embodiment wherein the indications comprise characterizations of extent of motion during time-interval/s, to identify time-windows in which an end-user was immobile, or less mobile, and which are unexpectedly or uncharacteristically long.

Embodiment 12. The system of any preceding embodiment wherein the externally generated scores comprise scores indicative of a respiratory disease such as but not limited to asthma or chronic obstructive pulmonary disease (COPD).

It is appreciated that endurance in long walks (e.g. prediction of the 6-minute walk test) may be derived from IMU data, and this endurance is indicative of possible respiratory disease, such as but not limited to asthma or chronic obstructive pulmonary disease (COPD).

Embodiment 13. The system of any preceding embodiment wherein at least one indication from among the indications derived from patient P's IMU data comprises a putative new diagnosis for patient P, such as a possible new stroke or possible new fall or possible new bout of influenza or possible new fracture.

It is appreciated that patient P may additionally have suffered other strokes or falls or bouts of illness or fractures in the past.

Embodiment 14. A computerized healthcare method providing healthcare remotely to end-users, the method comprising providing at least one instance of an interface uploading IMU data recorded by a healthcare providing organization O's end users' communication devices' accelerometers; and/or providing at least one instance of a processor which maps indications derived from the IMU data to care actions, where the care actions comprise at least one allocation of at least one organizational resource from among a set of available organizational resources which the organization allocates to its end-users or patients or members, thereby to reliably and efficiently allocate available organizational resources to at least one organization's patients.

Embodiment 15. A computer program product, comprising a non-transitory tangible computer readable medium having computer readable program code embodied therein, the computer readable program code adapted to be executed to implement a computerized healthcare method providing healthcare remotely to end-users, the method comprising providing at least one instance of an interface uploading IMU data recorded by a healthcare providing organization O's end users' communication devices' accelerometers; and/or providing at least one instance of a processor which maps indications derived from the IMU data to care actions, where the care actions comprise at least one allocation of at least one organizational resource from among a set of available organizational resources which the organization allocates to its end-users or patients or members, thereby to reliably and efficiently allocate available organizational resources to at least one organization's patients.

Also provided, excluding signals, is a computer program comprising computer program code means for performing any of the methods shown and described herein when the program is run on at least one computer; and a computer program product, comprising a typically non-transitory computer-usable or-readable medium e.g. non-transitory computer-usable or -readable storage medium, typically tangible, having a computer readable program code embodied therein, the computer readable program code adapted to be executed to implement any or all of the methods shown and described herein. The operations in accordance with the teachings herein may be performed by at least one computer specially constructed for the desired purposes, or a general-purpose computer specially configured for the desired purpose by at least one computer program stored in a typically non-transitory computer readable storage medium. The term “non-transitory” is used herein to exclude transitory, propagating signals or waves, but to otherwise include any volatile or non-volatile computer memory technology suitable to the application.

Any suitable processor/s, display and input means may be used to process, display, e.g., on a computer screen or other computer output device, store, and accept information such as information used by or generated by any of the methods and apparatus shown and described herein; the above processor/s, display and input means including computer programs, in accordance with all or any subset of the embodiments of the present invention. Any or all functionalities of the invention shown and described herein, such as but not limited to operations within flowcharts, may be performed by any one or more of: at least one conventional personal computer processor, workstation or other programmable device or computer or electronic computing device or processor, either general-purpose or specifically constructed, used for processing; a computer display screen and/or printer and/or speaker for displaying; machine-readable memory such as flash drives, optical disks, CDROMs, DVDs, BluRays, magnetic-optical discs or other discs; RAMs, ROMs, EPROMS, EEPROMs, magnetic or optical or other cards, for storing, and keyboard or mouse for accepting. Modules illustrated and described herein may include any one or combination or plurality of: a server, a data processor, a memory/computer storage, a communication interface (wireless (e.g., BLE) or wired (e.g., USB)), a computer program stored in memory/computer storage.

The term “process” as used above is intended to include any type of computation or manipulation or transformation of data represented as physical, e.g. electronic, phenomena which may occur or reside e.g. within registers and/or memories of at least one computer or processor. Use of nouns in singular form is not intended to be limiting; thus the term processor is intended to include a plurality of processing units which may be distributed or remote, the term server is intended to include plural typically interconnected modules running on plural respective servers, and so forth.

The above devices may communicate via any conventional wired or wireless digital communication means, e.g., via a wired or cellular telephone network, or a computer network such as the Internet.

The apparatus of the present invention may include, according to certain embodiments of the invention, machine readable memory containing or otherwise storing, a program of instructions, which, when executed by the machine, implements all or any subset of the apparatus, methods, features, and functionalities of the invention shown and described herein. Alternatively, or in addition, the apparatus of the present invention may include, according to certain embodiments of the invention, a program as above which may be written in any conventional programming language, and optionally a machine for executing the program, such as but not limited to a general-purpose computer which may optionally be configured or activated in accordance with the teachings of the present invention. Any of the teachings incorporated herein may, wherever suitable, operate on signals representative of physical objects or substances.

The embodiments referred to above, and other embodiments, are described in detail in the next section.

Any trademark occurring in the text or drawings is the property of its owner and occurs herein merely to explain or illustrate one example of how an embodiment of the invention may be implemented.

Unless stated otherwise, terms such as, “processing”, “computing”, “estimating”, “selecting”, “ranking”, “grading”, “calculating”, “determining”, “generating”, “reassessing”, “classifying”, “generating”, “producing”, “stereo-matching”, “registering”, “detecting”, “associating”, “superimposing”, “obtaining”, “providing”, “accessing”, “setting” or the like, refer to the action and/or processes of at least one computer/s or computing system/s, or processor/s or similar electronic computing device/s or circuitry, that manipulate and/or transform data which may be represented as physical, such as electronic, quantities, e.g., within the computing system's registers and/or memories, and/or may be provided on-the-fly, into other data which may be similarly represented as physical quantities within the computing system's memories, registers or other such information storage, transmission or display devices or may be provided to external factors e.g. via a suitable data network. The term “computer” should be broadly construed to cover any kind of electronic device with data processing capabilities, including, by way of non-limiting example, personal computers, servers, embedded cores, computing systems, communication devices, processors (e.g., digital signal processors (DSPs), microcontrollers, field programmable gate arrays (FPGAs), application specific integrated circuits (ASICs), etc.) and other electronic computing devices. Any reference to a computer, controller, or processor, is intended to include one or more hardware devices, e.g., chips, which may be co-located or remote from one another. Any controller or processor may, for example, comprise at least one CPU, DSP, FPGA or ASIC, suitably configured in accordance with the logic and functionalities described herein.

Any feature or logic or functionality described herein may be implemented by processor/s or controller/s configured as per the described feature or logic or functionality, even if the processor/s or controller/s are not specifically illustrated for simplicity. The controller or processor may be implemented in hardware, e.g., using one or more Application-Specific Integrated Circuits (ASICs) or Field-Programmable Gate Arrays (FPGAs) or may comprise a microprocessor that runs suitable software, or a combination of hardware and software elements.

The present invention may be described, merely for clarity, in terms of terminology specific to, or references to, particular programming languages, operating systems, browsers, system versions, individual products, protocols and the like. It will be appreciated that this terminology or such reference/s is intended to convey general principles of operation clearly and briefly, by way of example, and is not intended to limit the scope of the invention solely to a particular programming language, operating system, browser, system version, or individual product or protocol. Nonetheless, the disclosure of the standard or other professional literature defining the programming language, operating system, browser, system version, or individual product or protocol in question, is incorporated by reference herein in its entirety.

Elements separately listed herein need not be distinct components, and alternatively may be the same structure. A statement that an element or feature may exist is intended to include (a) embodiments in which the element or feature exists; (b) embodiments in which the element or feature does not exist; and (c) embodiments in which the element or feature exist selectably, e.g., a user may configure or select whether the element or feature does or does not exist.

Any suitable input device, such as but not limited to a sensor, may be used to generate or otherwise provide information received by the apparatus and methods shown and described herein. Any suitable output device or display may be used to display or output information generated by the apparatus and methods shown and described herein. Any suitable processor/s may be employed to compute or generate or route, or otherwise manipulate or process information as described herein and/or to perform functionalities described herein and/or to implement any engine, interface, or other system illustrated or described herein. Any suitable computerized data storage, e.g., computer memory, may be used to store information received by or generated by the systems shown and described herein. Functionalities shown and described herein may be divided between a server computer and a plurality of client computers. These or any other computerized components shown and described herein may communicate between themselves via a suitable computer network.

The system shown and described herein may include user interface/s e.g. as described herein, which may, for example, include all or any subset of: an interactive voice response interface, automated response tool, speech-to-text transcription system, automated digital or electronic interface having interactive visual components, web portal, visual interface loaded as web page/s or screen/s from server/s via communication network/s to a web browser or other application downloaded onto a user's device, automated speech-to-text conversion tool, including a front-end interface portion thereof and back-end logic interacting therewith. Thus, the term user interface or “UI” as used herein includes also the underlying logic which controls the data presented to the user, e.g., by the system display, and receives and processes and/or provides to other modules herein, data entered by a user, e.g., using her or his workstation/device.

### BRIEF DESCRIPTION OF THE DRAWINGS

Certain embodiments of the present invention are illustrated in the following drawings; in the block diagrams, arrows between modules may be implemented as APIs, and any suitable technology may be used for interconnecting functional components or modules illustrated herein in a suitable sequence or order, e.g., via a suitable API/Interface. For example, state of the art tools may be employed, such as but not limited to Apache Thrift and Avro which provide remote call support. Or, a standard communication protocol may be employed, such as but not limited to HTTP or MQTT, and may be combined with a standard data format, such as but not limited to JSON or XML. According to one embodiment, one of the modules may share a secure API with another module. Communication between modules may comply with any customized protocol or customized query language, or may comply with any conventional query language or protocol. Illustrated screenshots or wireframes may be generated by the app and/or dashboard of FIG. 1

FIG. 1 is a simplified block diagram illustration of an example system according to embodiments of the present invention.

FIG. 2a is an example home screen for the patient's facing app of FIG. 1;

FIG. 2b is a screen which presents plural (e.g. all available) mobility tests;

FIG. 3a is an example screen of a patient's progress, including a measurement summary screen;

FIG. 3b illustrates widgets (or any application or interface component);

FIG. 4 illustrates an example of an individual patient's screen which may be generated by the web-service of FIG. 1 and displayed on the dashboard of FIG. 1;

FIGS. 5a-5b pertain to an example review of a patient's indication that may be generated by the web-service of FIG. 1 and displayed on the dashboard of FIG. 1;

FIG. 6 is an example digital care service monitoring flow e.g. for the web service/server of FIG. 1;

FIGS. 7-9 illustrate workflows all or any subset of which may be supported by the system wherein all or any subset of illustrated operations may be provided in practice, in any suitable order e.g. as shown; specifically

FIG. 7 illustrates an example workflow for adjustment of assistive device (or orthotics and prosthetics) in the clinic setings and then monitoring patient adaptation to the device;

FIG. 8 illustrates—inpatient workflow (e.g. for patients hospitalized in a facility) followed by outpatient workflow; and

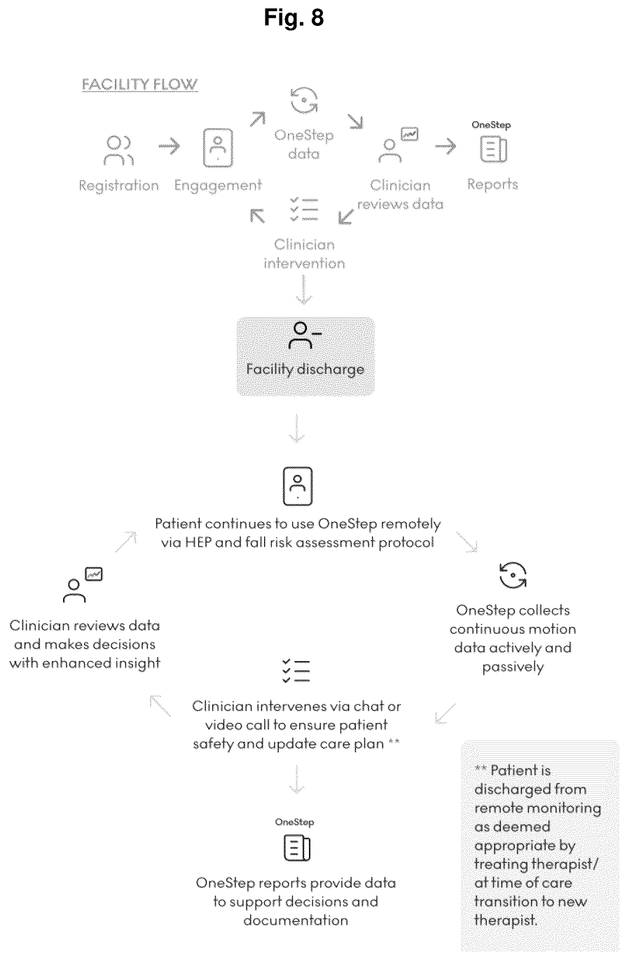

FIG. 9 illustrates outpatient workflow (only).

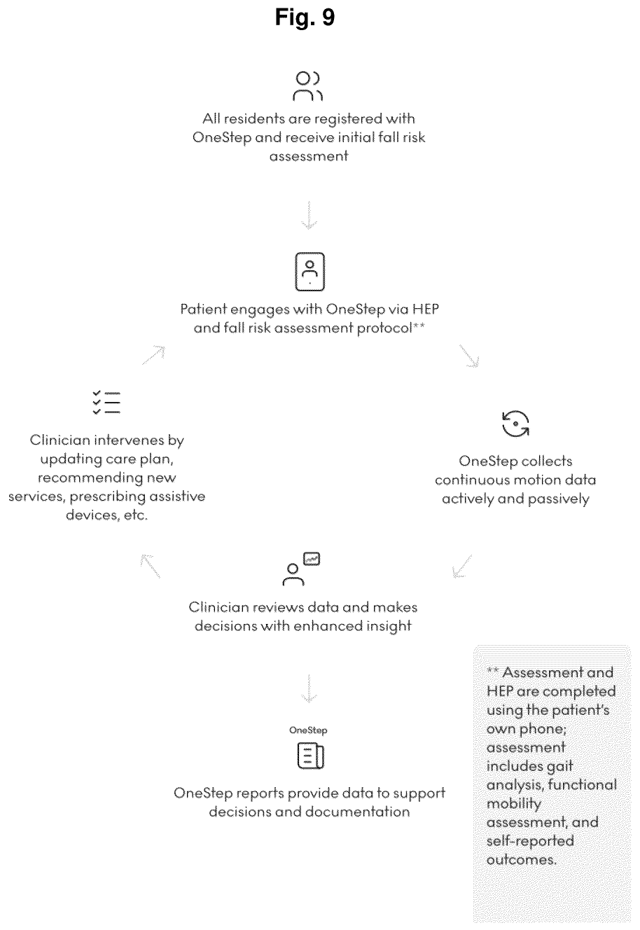

FIG. 10 illustrates a table useful in understanding certain embodiments.

FIG. 11 illustrates a protocol completion bar according to an embodiment of the present invention.

Methods and systems included in the scope of the present invention may include any subset or all of the functional blocks shown in the specifically illustrated implementations by way of example, in any suitable order, e.g., as shown. Flows may include all or any subset of the illustrated operations, suitably ordered, e.g., as shown. Tables herein may include all or any subset of the fields and/or records and/or cells and/or rows and/or columns described.

Computational, functional or logical components described and illustrated herein can be implemented in various forms, for example as hardware circuits, such as but not limited to custom VLSI circuits or gate arrays or programmable hardware devices such as but not limited to FPGAs, or as software program code stored on at least one tangible or intangible computer readable medium and executable by at least one processor, or any suitable combination thereof. A specific functional component may be formed by one particular sequence of software code, or by a plurality of such, which collectively act or behave or act as described herein with reference to the functional component in question. For example, the component may be distributed over several code sequences, such as but not limited to objects, procedures, functions, routines, and programs, and may originate from several computer files which typically operate synergistically.

Each functionality or method herein may be implemented in software (e.g. for execution on suitable processing hardware such as a microprocessor or digital signal processor), firmware, hardware (using any conventional hardware technology such as Integrated Circuit technology) or any combination thereof.

Functionality or operations stipulated as being software-implemented may alternatively be wholly or fully implemented by an equivalent hardware or firmware module, and vice-versa. Firmware implementing functionality described herein, if provided, may be held in any suitable memory device, and a suitable processing unit (aka processor) may be configured for executing firmware code. Alternatively, certain embodiments described herein may be implemented partly or exclusively in hardware, in which case all or any subset of the variables, parameters, and computations described herein may be in hardware.

Any module or functionality described herein may comprise a suitably configured hardware component or circuitry. Alternatively or in addition, modules or functionality described herein may be performed by a general purpose computer, or more generally by a suitable microprocessor, configured in accordance with methods shown and described herein, or any suitable subset, in any suitable order, of the operations included in such methods, or in accordance with methods known in the art.

Any logical functionality described herein may be implemented as a real time application, if, and as appropriate, and which may employ any suitable architectural option, such as but not limited to FPGA, ASIC, or DSP, or any suitable combination thereof.

Any hardware component mentioned herein may in fact include either one or more hardware devices, e.g., chips, which may be co-located or remote from one another.

Any method described herein is intended to include, within the scope of the embodiments of the present invention, also any software or computer program performing all or any subset of the method's operations, including a mobile application, platform or operating system, e.g., as stored in a medium, as well as combining the computer program with a hardware device to perform all or any subset of the operations of the method.

Data can be stored on one or more tangible or intangible computer readable media stored at one or more different locations, different network nodes or different storage devices at a single node or location.

It is appreciated that any computer data storage technology, including any type of storage or memory and any type of computer components and recording media that retain digital data used for computing for an interval of time, and any type of information retention technology, may be used to store the various data provided and employed herein. Suitable computer data storage or information retention apparatus may include apparatus which is primary, secondary, tertiary, or off-line; which is of any type or level or amount or category of volatility, differentiation, mutability, accessibility, addressability, capacity, performance and energy use; and which is based on any suitable technologies such as semiconductor, magnetic, optical, paper, and others.

### DETAILED DESCRIPTION OF CERTAIN EMBODIMENTS

Certain embodiments seek to provide a computerized healthcare system providing health care remotely to end-users, the system comprising at least one instance of an interface uploading mobility data recorded by a healthcare-providing organization O's end users' communication devices' accelerometers; and a processor which maps mobility-based indications to individual organizational resources from among a set of available organizational resources which the organization allocates to its end-users or patients or members (such as care actions performed within the organization and/or hardware assigned to end-users; it is appreciated that allocation of hardware to end-users may be considered a care action), thereby to reliably and efficiently assign available organizational resources to at least one organization's patients. The terms “IMU” and “accelerometer” may be interchanged herewithin.

The system may include plural instances of the interface and of the processor, for plural healthcare providing organizations O1, . . . ON and wherein the organizational resources, which at least one organization Oj allocates to its end-users or patients or members, includes referrals to outside organizations, other than organization Oj, which typically perform care actions not performed within the organization Oj.

Recording of mobility data is typically unobtrusive and/or frequent and/or continuous (e.g. does not require activation) and/or passive. The term continuous, as used herein, may, for example, refer to at least 1/day (for organizations which allocate resources daily), and/or whenever a new walk is measured. More generally, “continuous” may refer to whatever frequency an organization uses for its resource allocation.

The processor typically maps not only indications, but also patient information, to resources. Thus, typically, the processor's allocation of resources depends not only on the organizations' users' mobility-based indications, but also on information regarding these users other than such indications, aka “patient information”.

It is appreciated that the organizations are in effect the end-users of the system, however each organization itself has end-users to which the organization (e.g. hospital, health-care system, assisted living center, rehab center, clinic, and so forth) provides health care, typically by optimally allocating organizational resources, both human and hardware, to the organization's end users, based on mobility-based indications characterizing these users inter alia.

Indications may comprise diagnostics derived from raw mobility data generated by IMUs or accelerometers (where IMU and accelerometer may be interchanged herewithin. For example, low cadence is an example indication derived from raw mobility data. Another such indication is “balance instability”, or “gait speed is slower than most people of patient's age” or “gait slower than two weeks ago”. An example care action is a given daily physical therapy care program, which may be suited for patients with a certain type of patient information and who also have over-threshold (aka too high) balance instability. Patient info may include test results, e.g. an electrocardiogram or a bone scan or a blood test, or hospital release papers or known diagnoses such as diabetes or moderate dementia, or basic data such as age, height, gender, or any other data pertaining to an end-user which is stored in relation to the end-user in the system and/or in the organizational end-user's data repository.

An indication may for example comprise a symptom (e.g. “limps”, “respiratory distress”) or a diagnosis (e.g. “Parkinson's disease”).

An organization e.g. HMO or hospital may relate an indication and, typically, a type or category of care (e.g. care context: outpatient vs inpatient), to a care action (or a recommendation for a care action).

The system herein may generate mobility measures e.g. gait speed, cadence or other gait parameters including composite measures (combinations of mobility measures) and indications may be defined over mobility measure/s e.g. “dramatic change in gait speed”, “cadence lower than reference norms”, “fast trend of recovery”, “fatigue”). Indications may be functions of mobility measures and norms. Indications may be correlated to, or may predict “pro” aka patient-reported outcomes, or other such condition-specific indexes/scores. In COPD (Chronic Obstructive Pulmonary Disease) for example, fatigue may be operationalized or measured as mobility decline during a long walk. Example patient-reported outcomes are SGRQ=St. George's Respiratory, UPDRS, PDQ, EDSS (by neurologist examination e.g. for MS), PDDS, LEFS for lower body functionality. Predictions of the above may be derived from (ultimately, for example accelerometer raw data e.g. using any embodiment herein or any embodiment described in Applicant's patent pending technology as described in any co-owned patent document mentioned herein, the disclosures of all of which are hereby incorporated by reference.

It is appreciated that mobility measures may be used to assess or estimate a standard outcome measure, e.g. by training a classifier to predict standard outcome measures from IMU raw data or from mobility measures (or gait parameters) derived from IMU raw data. It is appreciated that plural values for a given gait parameter e.g. cadence or gait velocity, time-stamped with plural different time-stamps respectively, may be included in the mobility measures used to predict standard outcomes. If no gait parameter value is available for a given time t sought to be included in the predicting mobility measures, e.g. because t represents a date on which no passive walks are available, the value may be interpolated based on values of the same gait parameter, with time-stamps other than t, which are available in the system (dates other than t on which passive walks were available, e.g.). Standard outcome measures may for example include PDQ scores and/or UPDRS scores and/or multiple sclerosis scores such as clinician-assigned EDSS scores and/or PDDS scores and/or other PROs aka patient questionnaires and/or respiratory SGRQ and/or clinical observations, which may even be binary e.g. “shuffling observed” or “limping observed” (observed e.g. by clinician and/or self-reported e.g. by patient and/or carer) as opposed to no such gait having been observed/self-reported. A model e.g. classifier may be trained to predict, say, “shuffling observed” or “limping observed”, using training data which includes mobility measures in the system, which preceded clinic visits entered into the system, for N (say 50) system users. Assuming these clinic visits resulted in clinician observations entered into the system, these observations may be used as labels at the training stage; some labels (assigned to some of the N system users) typically report “shuffling observed” or “limping observed” as opposed to other labels (assigned to others of the N system users) in which no such gait was observed and/or in which the system prompted the clinician to report whether shuffling (say) was or was not observed and for some of the system users, shuffling was reported to have been observed, whereas for others of the system users, shuffling was explicitly ruled out via the system.

A composite measure based on mobility measures that correlates with respiratory measures or clinical outcomes such as respiratory rate, Oxygen Saturation (SpO₂), may be defined as a respiratory status indication.

Typically, once a specific organization onboards as an organizational end-user of the system herein, the implementation team configures rules according to the organization's possibly changing needs. For example, if it is an organization that needs to prioritize patients for an advanced care/skilled care program such as daily physical therapy care, this organizational end-user may have rules that recommend a referral for a specific care program for patients with higher balance instability. Domain experts (clinicians) typically define rules; technical setup of the rules may be performed by the organizational user's technical team (e.g. IT/customer support/engineers). Mapping between indications and care actions is typically fully determined by the system's rules.

A “rule” may indicate, for example, that phone accelerometer data x could be indicative of diagnosis y which warrants care-action z.

Typically, rules mapping (or associating in memory) certain values or levels of certain mobility measure/s or gait parameters, or certain indications to certain care actions, are generated manually, initially. Subsequently, the system may collect care actions that were acted upon, and corresponding mobility measures and indications associated with these care actions and, accordingly, may learn better mapping between indications and care actions, which may, with time, replace or augment (e.g. as an automatically generated recommended rule) the original, manually generated rules that originally were used by the system to map certain indications to certain care actions.

Thus, according to certain embodiments, the system is configured to learn the relationship between indications and care actions (interventions) executed and, accordingly, to recommend care actions for indications in the future, in an ongoing (online) learning process. typically the system machine-learns the rules e.g. what care actions do the rules defined to date assign to various indications and then, instead of having to continue defining rules, the system can recommend a care action for each indication. alternatively or in addition, the system may receive outcomes of care actions for certain indications and may learn which care actions have better outcomes.

Typically, each organizational user defines care actions; technical setup of the care actions may be performed by the organizational user's technical team (e.g. IT/customer support/engineers). A health-care system is an example of an “organizational user”; other examples are hospitals, rehab centers, and clinics, or networks of the same. It is appreciated that hierarchies of organizational users may be defined, e.g. each branch of a network may be an organizational user deployed below the network as a whole, which may also be defined as an organizational user.

A given care action may comprise one or more organizational resources allocated for the patient which may include a service/action, or may include allocation of goods/resources, to an organization's “end-users” or members. A care-action defined by an organizational user may be “allocate a respirator (or wheelchair) to end-user x”. One care-action may be “do nothing”; rules may assign this care-action to end-users who do not appear to require any intervention from the organizational user.

Any suitable entity using the system may be authorized to define each organization's set of care actions, e.g. the organizational user's IT department together with admin personnel who coordinate services, internal and external (via referrals to other organizations) provided to the organizational user's end users, and therefore may conveniently generate a set of services (“care actions”) the organizational user provides.

Defining a set of care actions may be done initially when an organization first joins the system as an organizational end-user, and may be updated e.g. each time a given organizational user decides to add certain services which were not available previously, or to discontinue certain services which were found to be harmful or insufficiently cost-effective.

Certain care actions may comprise allocation of at least one organizational resource to a given member or patient or end-user (typically allocating from among a typically finite (at any given point in time) set of available organizational resources which the organization allocates to its end-users or patients or members). The resources may include care services performed within the organization, e.g. assignment of organization hardware to end-users, or administering treatment to end-users even without using any hardware, or conducting a session to end-users even without using any hardware, e.g. for consultation. It is appreciated that services are typically finite because the organization has a finite number of employees who work a finite number of hours. One resource may be referral to an external organization which itself performs care services; each of these care services may in turn include assignment of the external organization's hardware (of which there is again a finite set at any given time) to end-users). Thus, a care-action by a first organization, e.g. an Israeli sick-fund, may comprise “referral to organization y”, e.g. rehabilitation facility or geriatric hospital, or a mental health care facility.

A given “care action” aka “intervention” may inter alia include a session of treatment and/or consultation and/or allocation of hardware e.g. “wheelchair”, “home oxygen tank” and/or allocation of a referral to an external organization.

Example “care actions” include, say, referring an end-user to a mobility specialist to recover in ambulation independence, or monitoring an end-user aka patient to prevent further stroke events, typically using a defined monitoring protocol and defined hardware.

Once an end-user or patient is referred to a geriatric hospital (say), this geriatric hospital may also have the platform subject of embodiment/s herein, and they may have certain internal care actions e.g. assignment of respiratory devices to patients. It is appreciated that some care actions are available in one organization, but not in another, e.g. organization1 may refer a patient to organization2 because organization2 has respiration as one of its care actions, and organization1 does not.

Typically, raw accelerometer or IMU data is processed, e.g. by conventional gait analysis, to yield mobility measures aka mobility data e.g. gait parameters such as gait speed, cadence, other spatiotemporal parameters, kinematics parameters, kinetics parameters, sitting time, and standing time.

Mobility measures include gait parameters (which may be derived from raw IMU data) and/or measures of mobility which do not pertain to gait/walking e.g. sitting time, turning speed.

“Indications” may comprise combinations of mobility measures e.g. combinations of a given end-user's longitudinal mobility measures (mobility measures over time), which may seek to correlate with or predict a clinical outcome (e.g. PRO aka patient reported outcome, or any other clinical assessment, index, etc.). “Outcomes” or “clinical outcomes” as used herein may include PROs, clinician assessments, lab results and markers, disease-specific indexes, reported falls, etc.; any of these may be predicted or estimated by a suitably defined indication, according to embodiments herein.

Example “indications” include a level of fall risk (e.g. severe/high/mid/low), a frailty indication, a level of audio impairment, a level or binary indication of presence of dyspnea, a level or binary indication of presence of fatigue, or a prediction/estimate of an outcome e.g. as described above. Indications may be derived from IMU data.

Indications may, for example, stipulate a level e.g. high/low/normal for any of the following: fall risk (e.g. as described herein), cognitive impairment, post-op recovery, progression of Parkinson's Disease, mobility affected by medication, adaptation to assistive devices or prosthetics, visual impairment, diabetic foot status, back chronic pain, spine pain mobility status, functional status of patients treated with chemotherapy, and clinical depression.

Another example of an indication is “mobility decline during a long walk” which may be regarded as a fatigue parameter.

An indication may stipulate a level of severity, functional assessment, progression of recovery, or deterioration in any medical and health-related condition at a specific point on the patient's timeline. For example, a fall risk indication of severe risk of fall indicates that a patient is assessed with a severe risk of falling on a specific date or period of time. Thus, an indication is a function of a patient's timeline at a specified time. Indication may compare the patient's timeline and data to the patient's relevant norms or standards, e.g., an indication of how fast or slow this patient recovers from a total hip replacement, compared to the recovery benchmark of a population of the same age and gender post-total hip replacement surgery. Typically, each indication has a level, e.g., positive, neutral, and negative, indicating whether it is a good or bad indication; an indication may be compared to another indication of the same patient, the same kind of indication but at another time specified, or another patient's indication of the same kind, and even between different kinds of indications. This allows for the sorting and segmenting of patients by indications. One way to implement this is to provide an indication with a numerical score, which may be presented in the clinician dashboard and the patient app. An indication may be nested with other indications. For example, a cognitive decline indication may be defined by comparing two consecutive cognitive impairment indications. An explanation of a nested indication is recursive.

According to embodiments, the system herein may be taught to derive “mobility decline during a long walk” from accelerometer raw data, e.g. by training a classifier using labels generated by experimental subjects with COPD aka Chronic Obstructive Pulmonary Disease, who may rate their fatigue after a given long walk that has been measured, as either “high” “medium” or “low”. It is appreciated that it may be possible to conclude that their gait speed decreases or asymmetry increases during a standard 6-min-walk test. The system may predict 6mwt performance from shorter walks (and/or from plural passive/background walks collected over, say, a week). The system may indicate in 6mwt when a significant change in gait occurs, e.g. “the patient's gait changed dramatically after 90 seconds,” then a week after perhaps this may occur “after 360 seconds,” whereas later on the patient may maintain the same speed with the same gait quality for the entire 6mwt. A fatigue indication for example, may enhance a digital care system for managing COPD, heart failure, and other chronic conditions in outpatients (in the community). This may for example enable continuous monitoring and.or proactive disease management, such as prompting further in-person assessments (e.g. EKG, installing a Holter) or hospitalization as appropriate.

It is appreciated that the above mobility-based indications aka accelerometer-measurable indicators (e.g. “mobility decline during a long walk”) are merely examples.

An example “mobility measure” may be characterizations of extent of motion over a period of time, e.g. to catch a time-window of 1 or 2 hours (say) in which an end-user did not move at all. An example of an indication may be the level of physical activity behavior (low/medium/high e.g.), which may be derived by combining the “extent of motion” mobility measure with a “level of cadence over past week” (e.g. slow/fast).

Indications may be mapped into care actions. This mapping may be determined by domain expert rules rather than by ML. The terms mobility measure, motion parameter, and gait parameter, may be interchanged herewithin, and, similarly, the term caregiver, clinician, physical therapist, occupational therapist, clinic manager, and even organization (given that these professionals are typically acting for an organization), may be interchanged herewithin.

It is appreciated that the system may store system data on a cloud e.g. raw data, mobility measures, indications, care actions/recommendations.

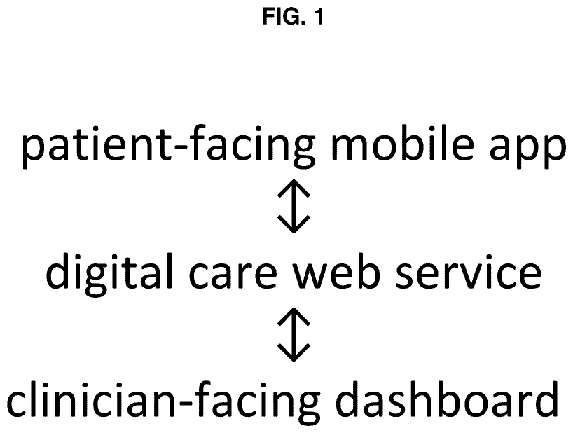

An example system is shown in FIG. 1; all or any subset of its processing units shown may be provided in practice (patient-facing mobile app and/or a clinician-facing dashboard and/or a digital care web service that typically communicates with the patient app and the clinician dashboard). More generally, any device/s and app/s connected via a server may be used. For simplicity, the organization served by the system is shown, in various drawings herein, to provide care actions within the field of physiotherapy only although this is not intended to be limiting.

It is appreciated that plural care actions may be nested within or hierarchically below another care action. Thus, if an organization provides a wide variety of care actions, including but not limited to physiotherapy, a higher level care action may be “provide physiotherapy for fracture” (or any other category of therapy) and, in addition, plural care actions within the realm of, or below, say, “provide post-stroke physiotherapy” may be defined, e.g. calling the patient undergoing physiotherapy to an in-person meeting, changing the exercise plan, asking the patient to take additional measurements or to undergo standard tests or to fill out a PRO, discharging the patient from treatment, or extending the treatment. Administration of exercises may also be defined as care actions below, for example “provide physiotherapy”:
1. Administer Obstacle course
2. Undergo Alternate fast-slow steps
3. Undergo Functional STS (sit-to-stand) transfer training

According to certain embodiments, the system herein e.g. web-service of FIG. 1 generates indication alerts that contain information about the patient's status and progress with relation to visually impairment on any given date. As input, the system takes into account the structured longitudinal patient data. This data contains spatiotemporal gait parameters measured with OneStep's mobile app which has gait assessment functionality (including but not limited to parameters such as velocity; step and stride length; double support, single support, and stance; measures of asymmetry such as stance asymmetry and step length asymmetry; and measures of variability such as cadence variability and velocity variability). It also contains results of standard tests (such as timed up and go and sit-to-stand tests, as well as patient reported outcomes. It may further contain kinetic and kinematic measures of gait e.g. as measured with OneStep's commercially available smartphone-based technology. From this data, the system assesses the patient's status based on both comparison to established norms and values found within the literature of research on visually impairment, and on the data collected by OneStep of patients with visual impairment. This status may contain information about the level of improvement and/or decline compared to a baseline point in time, and explain the information that went into the decision-making process for generating the status indication. Such indications may also contain suggested therapeutic interventions tailored to the patient based on the longitudinal data of that patient.

It is appreciated that any suitable information may be generated by the web service server of FIG. 1 and may be presented to the patient (via the app of FIG. 1 e.g.) and/or to the clinician (via the dashboard of FIG. 1 e.g.). For example:

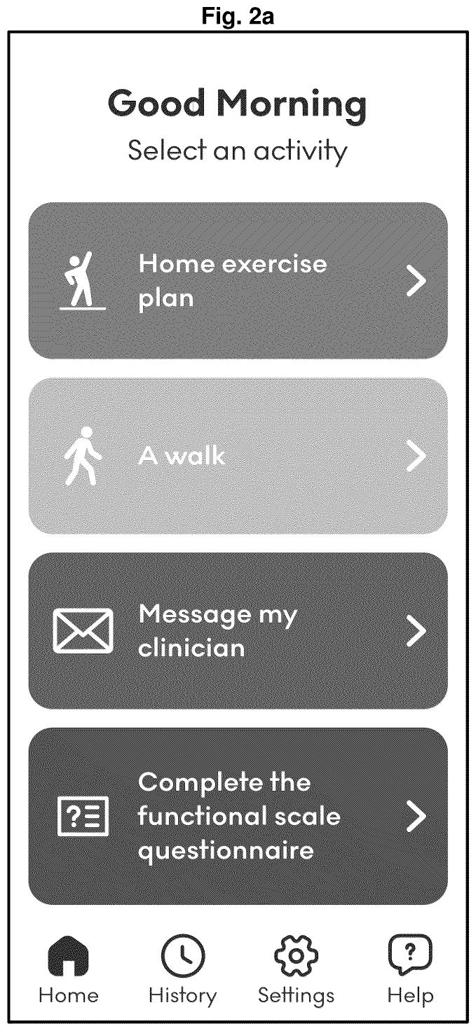

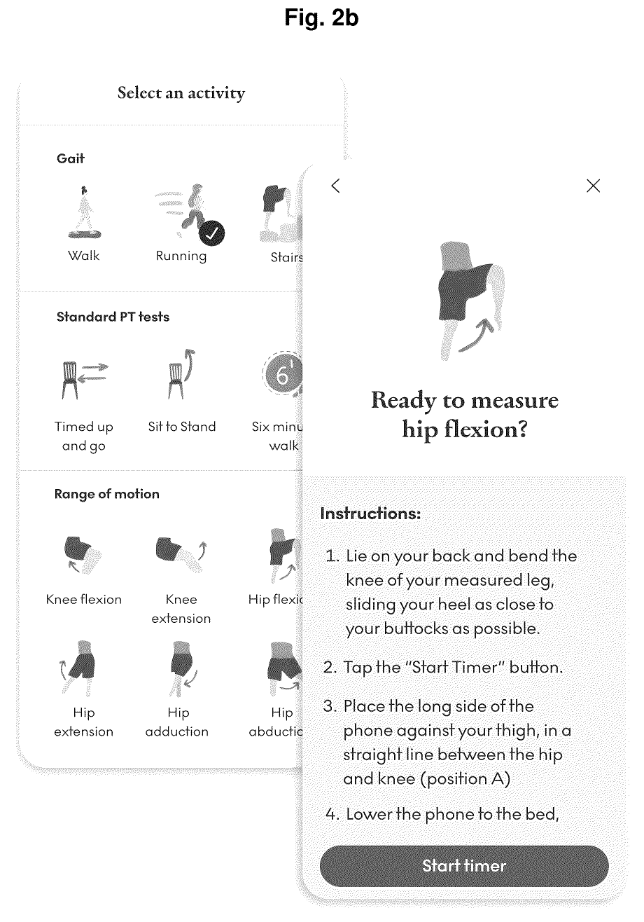

FIG. 2a is an example home screen for the patient's facing app of FIG. 1 which typically provides call to actions (for the patient). FIG. 2b is a screen which presents plural (e.g. all available) mobility tests which may be taken and/or measurements for a given user/patient.

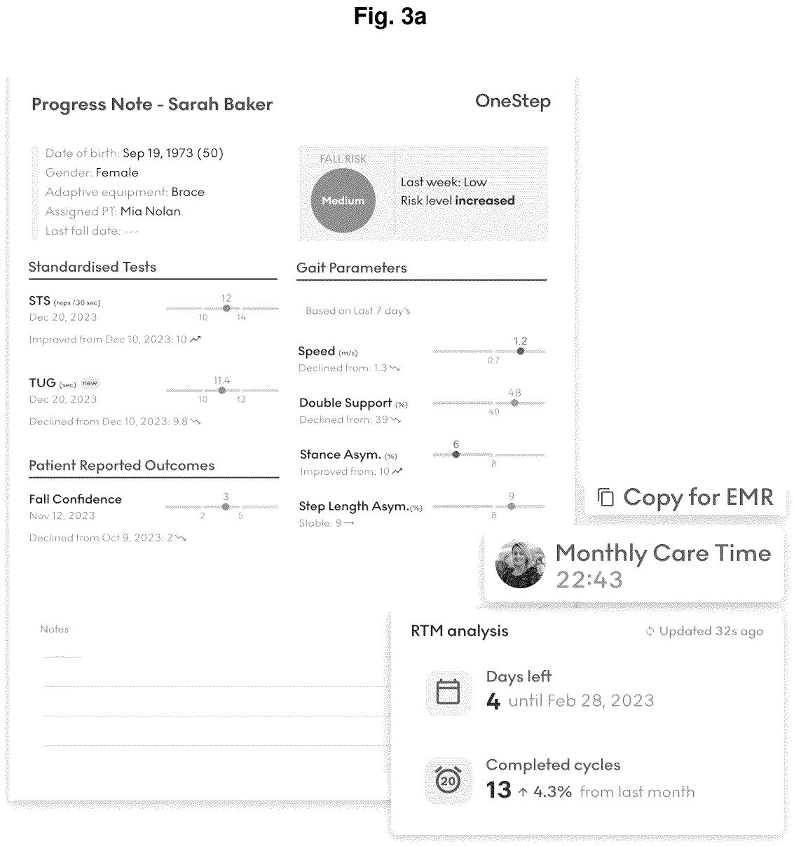

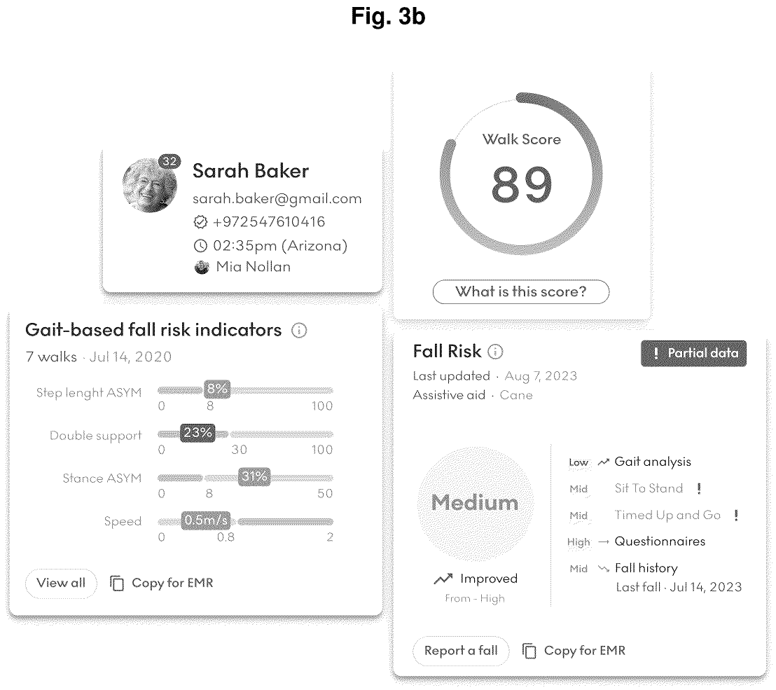

FIG. 3a is an example of a patient's progress note which may comprise a pdf which summarizes the patient's status and may comprise the patient's summary of mobility measures and indications, e.g. for an electronic medical records system in data communication with the system of FIG. 1. FIG. 3b illustrates widgets (or any application or interface component), that present indications such as but not limited to all or any subset of: walk score (composite gait quality score aggregating plural gait parameters/measures into a single score), Fall risk (composite score combining gait parameter/s, standard tests, fall history and patient-reported outcomes); and a break-down of gait measure contributors to fall risk assessment. The system may also accumulate and present remote treatment time spent on each patient, e.g. for documentation purposes.

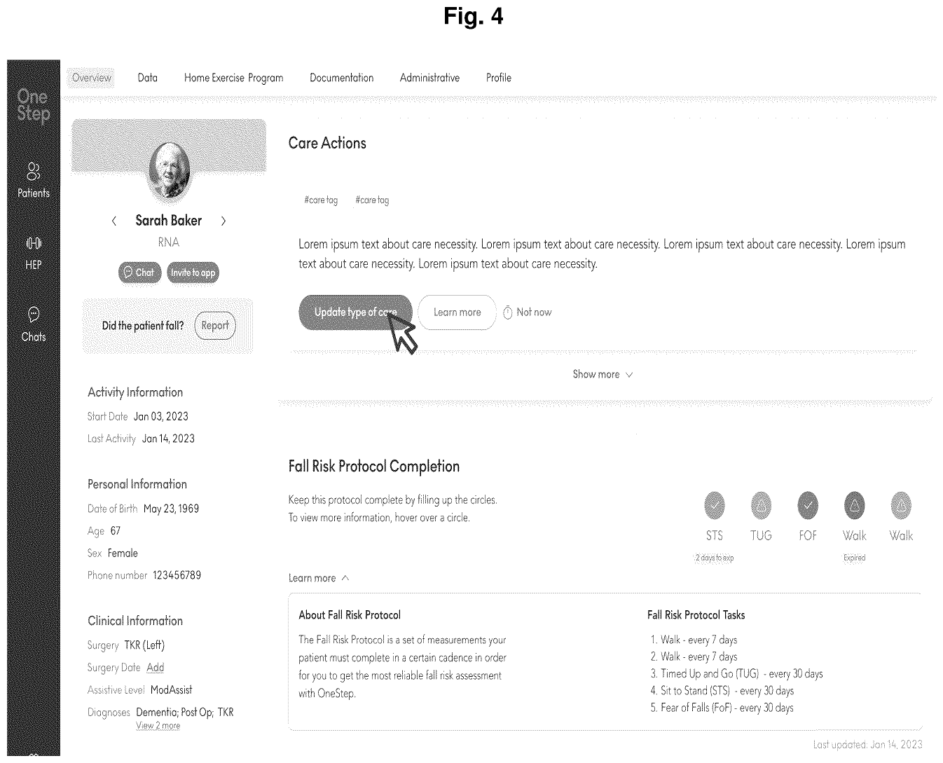

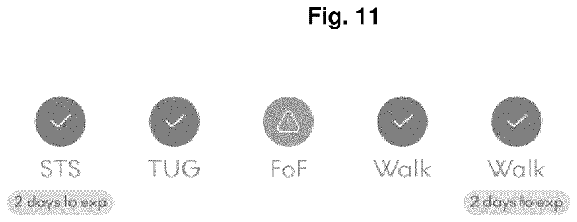

FIG. 4 illustrates an example of an individual patient's screen which may be generated by the web-service of FIG. 1 and displayed on the dashboard of FIG. 1. Typically, a clinician end-user of the system of FIG. 1 may consume the individual patient's care actions from this page that summarizes the patients status, mobility measures, and indications. The patient's screen, aka patient page, typically includes a protocol completion bar, indicating measurement/s a given patient should complete in order to provide enough data for computing indications more accurately. FIG. 11 is an example of this bar; in the illustrated example, as indicated by V's, almost all measurements have been completed (although 2 measurements, STS and walk, are about to expire; it is appreciated that any suitable CRITERION FOR EXPIRY may be defined such as “MORE THAN 2 months OLD”. However, the Fear of Falls (FoF) questionnaire as indicated by a warning triangle, has expired (or was never completed) thus needs to be completed. It is appreciated that the system may be configured to ensure or to strive to have plural non-expired walks available at any time since plural samples better represent the patient's behavior as opposed to only a single sample and/or since plural samples enable data quantifying variation/repeatability between walks to be derived hence available to the system.

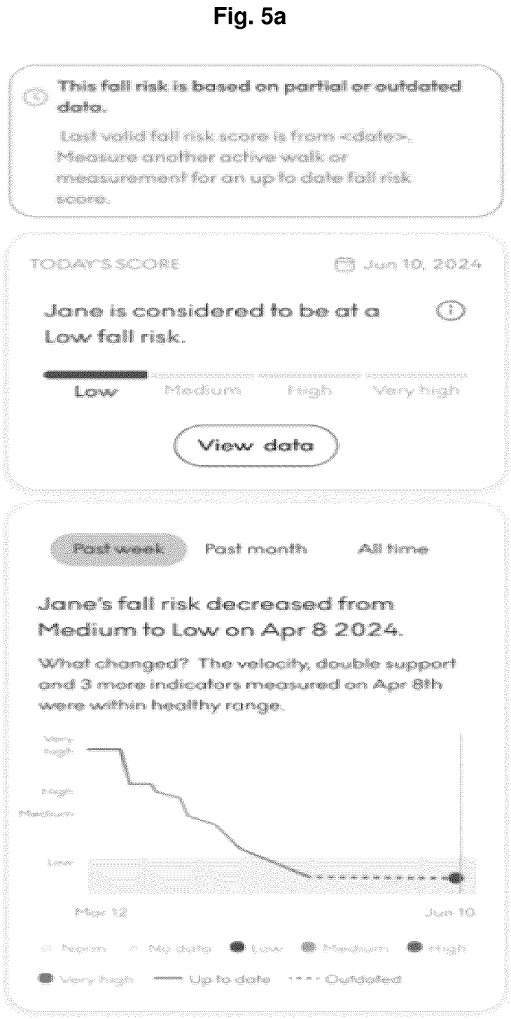

FIGS. 5a-5b pertain to an example review of a patient's indication that may be generated by the web-service of FIG. 1 and displayed on the dashboard of FIG. 1. FIG. 5a illustrates an example fall risk trend widget. FIG. 5b illustrates a fall risk indicator breakdown, typically including key mobility measure contributors. It is appreciated that a continuous e.g. daily fall risk indication may be presented to a patient's carer whether individual or an organization.

The system herein e.g. the clinician-facing dashboard, may provide triaging or prioritization functionality. For example, given a group of patients, there may be various options to manage care attention, and to select the patients with higher needs e.g. all or any subset of:
1 Segmentation—for example, dividing the patient into subgroups of patients with severe risk of falls, high risk of falls, and moderate and low-risk levels. Segmentation is typically available for any indication since typically, every indication has levels of severity.
2. Prioritization of patients—e.g. a sorted list of patients ordered from patients at higher risk to lower
3. Classification.

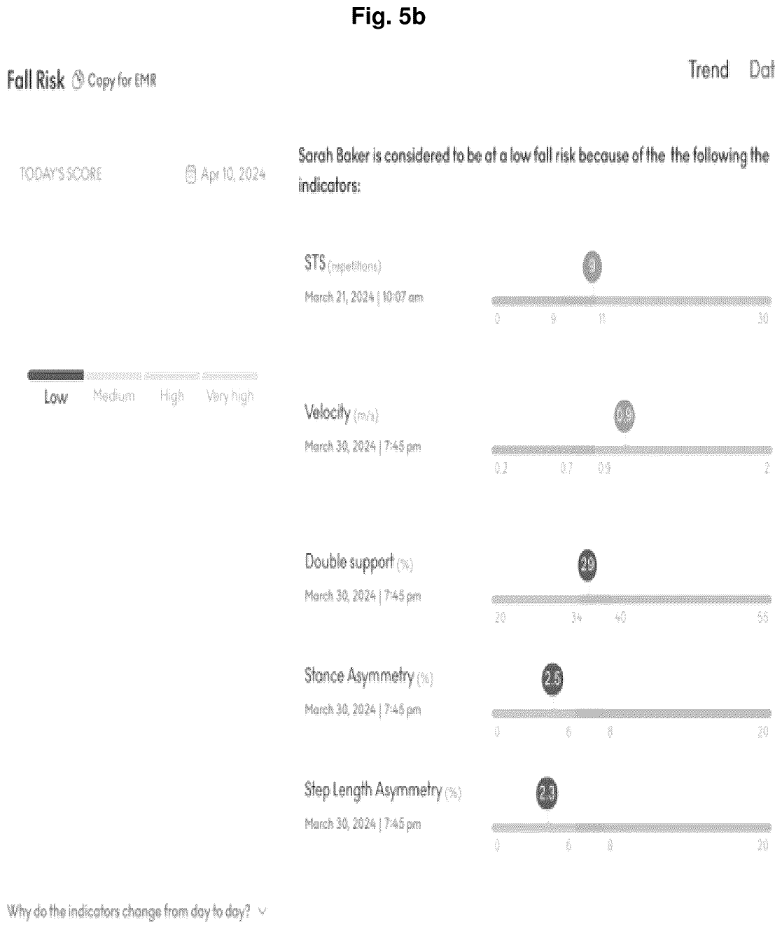

FIG. 6 is an example digital care service monitoring flow (e.g. for the web service/server of FIG. 1). All or any subset of the following operations may be provided, in any suitable order e.g. as shown: Start assessment, gather all patient's information e.g. all or any subset of mobility information, norms, subjective information, clinical events and HEP (home exercise plan), and assessment of the patient's current status e.g. all or any subset of fall risk status, change in mobility vs. onboarding, surgery, last month . . . , comparison to reference population, recovery pace, data re cognitive decline, visual deterioration, etc.

Referring again to the system of FIG. 1, embodiments of the three illustrated system components are now described in detail.
1. A patient-facing mobile app—this component enables the system to collect data from the patient, as well as communicate back to the patient to provide clinical value and encourage the patient to be engaged and adhere to their care program.
2. A clinician dashboard—this component allows the clinician to take different actions of care based on the patient's data for the individual patient. Moreover, it enables the management of clinical resources such as clinician attention and triage of patients in the scope of a clinic having a group of patients. Furthermore, a clinic manager or an organizational manager may compare different clinics in terms of mobility assessment of their patients in order to make organizational decisions. All of any subset of these three levels of data-driven decisions may be supported by the clinician dashboard.
3. A digital care web service—this component is responsible for communication with the two other components, which enables their functionalities. In addition, it is responsible for the analysis of the data collected from the patient app and the actions taken in the clinician dashboard to provide new calls to action continuously.

The system typically comprises clinician tools, e.g. care actions that depend on the mobility assessment of the individual and the plural patients. The system may utilize motion analysis including gait and mobility measurement using a mobile app on a single device with IMU, e.g. for general workflows of the clinician in their dashboard and the monitoring mechanism in the digital service component, all as described below. Any suitable technology may be employed to facilitate the activities of the clinician and the patient in the app and the dashboard, such as but not limited to those described below, which use the patient app and/or monitoring flow of FIG. 6 and/or indications and/or care actions described herein.

Typically, the patient app serves as an endpoint of the system on the patient's side. It enables the system to collect data from the patient, and the patients to understand their status over the treatment, communicate with their clinician, and consume their clinician's treatment program at home. The following aspects of the patient's app: patient data collection, patient status reflection, and communication with the clinician and home program may all be provided, or, alternatively, only a subset thereof may be provided. These three aspects comprise a crucial part of the system's operation since it depends on patient data, and reflection of the patient's status increases engagement, increasing the data available for the system. Moreover, communication and the home program increase patient engagement as well, and comprise some of the clinician's care actions of communication and prescribing a home program, which is the purpose of the system as a whole. As mentioned, example care actions are described below in the context of the description below of the clinician dashboard.

### Aspect1—Collection of Patient Data

The patient app of FIG. 1 may collect all or any subset of the following three kinds of patient data (objective, tests, subjective):
1. Objective gait analysis which may yield all or any subset of the following measures:
   a. Spatiotemporal analysis: evaluation of gait properties or gait parameters such as gait cycle time, cadence, gait speed, stride length, right and left step length, base width, right and left single support and stance time and percentage, double support time and percentage.
      Described in US 2020/0289027 A1
   b. Kinematics: including any of the lower body joint angles over the gait cycle Described in Ser. No. 17/666,180
   c. Kinetic: evaluation of gait properties related to torques and forces such as ground forces and center of feet pressure over the gait cycle, and the analysis of its sway over the gait cycle
   d. Variability: the deviation of any of the gait properties above between different strides over the same walk. may be processed in two forms, first aggregated as the standard deviation of each value. Second-as a sequence of the differences in values over time.
2. Mobility tests include an objective assessment of standard functional tests, for example all or any subset of:
   a. TUG—www.physio-pedia.com/Timed_Up_and_Go_Test_(TUG)
   b. Sit to stand—www.physio-pedia.com/30_Seconds_Sit_To_Stand_Test
   c. 6-minute walk test—www.physio-pedia.com/Six_Minute_Walk_Test_/_6_Minute_Walk_Test
   d. Rhomberg balance test—www.physio-pedia.com/Romberg_Test
   e. Four square step test—www.physio-pedia.com/Four_Square_Step_Test
   f. The 4-Stage Balance Test—www.physio-pedia.com/The_4-Stage_Balance_Test
   g. Reaction time in walking—the user is asked to walk back and forth and make U-turns (right or left) according to cues signaled by the device. The time between the cues' timestamps and the corresponding turns timestamps (extracted as described in US 2022/0111257) is then averaged and evaluated as the user's reaction time.
3. Subjective reporting of the patient includes questionnaires and patient-reported outcome measures such as all or any subset of:
   a. Pain rating—www.physio-pedia.com/Numeric_Pain_Rating_Scale
   b. Quick DASH—www.physio-pedia.com/DASH_Outcome_Measure
   c. NDI questionnaire—www.physio-pedia.com/Neck_Pain_and_Disability_Scale
   d. The Falls Efficacy Scale—www.physio-pedia.com/Falls_Efficacy_Scale _-_International_(FES-I)
   e. FABQ questionnaire—www.physio-pedia.com/Fear_Avoidance_Belief_Questionnaire
   f. Fear of Falls—www.physio-pedia.com/Falls_Efficacy_Scale _-_ International_(FES-I)
   g. PlusM—olc.oandp.org/resource/docs/aaop/1513371091260/PLUS-M.pdf
   h. AMPPro—www.oandp.com/opie/ampdownloads/assets/pdfs/amppro.pdf
   i. The Lower Extremity Functional Scale—www.physio-pedia.com/Lower_Extremity_Functional_Scale_(LEFS)
   j. The WOMAC Osteoarthritis Index—www.physio-pedia.com/WOMAC_Osteoarthritis_Index
   k. The Oswestry questionnaire—www.physio-pedia.com/Oswestry_Disability_Index
   l. www.physio-pedia.com/36-Item_Short_Form_Survey_(SF-36)

Embodiments of all of the above all three functionalities are described in co-owned US 2024/0008766 entitled “System, Method and Computer Program Product For Processing a Mobile Phone User's Condition”, the disclosure of which is hereby incorporated by reference in its entirety.

While all three data categories may be intentionally collected by the patient within the app workflow, the app typically allows the first category of gait analysis to be collected passively when the patient carries the mobile device throughout the day (as exists in prior art). Typically, the cadence of active measurement depends on the type of care and the condition of the patients, and how frequently it is expected to change. In general, patients should measure their walks, say, once a week or more when their condition changes rapidly, and once every two weeks when conditions change at a slower rate.

Reflection of patient status is typically provided by the system. The patient's status typically depends on the patient's type of care, defined in the patient's registration, and may be modified at a later stage (more details follow in the clinician dashboard's description). The status typically represents all or any subset of: levels of severity, functional assessment, progression of recovery, or deterioration in any medical and health-related condition, known as indications. For example, patients in a skilled nursing program type of care may have a fall risk indication. Patients with a degenerative retina disease may have a mobility indication affected by visual impairment. In addition, patients may have several indications. For instance, skilled nursing patients may also have a cognitive impairment indication.

Any suitable method may be used to generate a level (e.g. high/mid/low) of fall risk. For example, fall risk may be computed as a sum of factors, each factor being a combination e.g. multiplicative product of:
a score (e.g. score/s for some or all questions in FoF—fear of falls questionnaire, TUG—time up and go score, STS—sit to stand score) or gait parameter (e.g. an asymmetry parameter e.g. stance asymmetry, step length asymmetry, or double support or velocity) or other typically ordinal factor e.g. fall history; and/or
a recency weight assigned to the score or gait parameter (high if recent, low otherwise); and/or
a clinical importance weighting parameter, e.g. if all of the above scores and parameters are used (thus the fall risk is computed as a sum of plural factors) then all may be weighted as equally clinically important other than fall history which may be weighted higher, whereas confidence about falling (2^{nd} question from FoF) may be weighted lower.

The patient's status is shown on the app's home screen and may also be presented after every measurement and patient report, e.g. in a measurement summary screen.

The purpose of showing the status to the patient is to encourage the patient to adhere to the care program and provide more data to improve and confirm the status assessment. The status indications may be explained to the patient using the data collected. FIG. 4 presents a fall risk indication and presents the main contributors and the contributing factors, based on the previous data collected and measured. Moreover, different indication levels usually come with different insights and recommendations, such as performing a follow-up test, having a physical appointment with the clinician, and modifying the home exercise program. FIG. 4 also presents recommendations for the home exercise program. The clinician customizes these recommendations based on the different levels of a condition's status, constituting care actions presented to the patients.

Indications may include any of the following: fall risk, cognitive impairment, post-op recovery, progression of Parkinson's Disease, mobility affected by medication, adaptation to assistive devices or prosthetics, visual impairment, diabetic foot status, back chronic pain, spine pain mobility status, functional status of patients treated with chemotherapy, and clinical depression.

Typically, the system supports communication with the clinician and home exercise program.

The home exercise program aka HEP assigned to a patient may be used to assign a customized exercise plan to patients to continue their rehabilitation process at home, as well as a data point collection for Remote Therapeutic Monitoring (RTM).

As shown in FIG. 6, each patient with a home exercise program aka HEP gets a personal exercise program (may be a daily exercise plan), which includes exercises personally tailored and customized from the exercise library. The exercise library includes hundreds of different exercises recorded and featured organized by categories such as upper/lower body, health condition, surgery type, gait parameter, and more. Examples of such exercises are Alternating Leg Taps on Step, Forearm Side Plank, Half Tree Pose. Every exercise includes a video with voice instruction, written instructions for patients to read, and any equipment the patient may need, such as a chair, pillow, or mat.

Patients are assigned a template exercise program based on their diagnosis. The app may give patients exercise reminders, as prescribed by their clinician. When the patient completes an exercise day, the app allows the patient to progress to the next day and notifies the clinician that an exercise session has been completed. Throughout their rehabilitation journey, patients may reach out and contact their clinician e.v. viat chat. They may comment on exercises, ask questions, or give updates. Easy access to chat in the app allows for ongoing communication during the patient's recovery journey.

The clinician dashboard provides the clinician with administrative tools or care actions, triaging tools or care actions which may be defined over sets of plural patients, and care actions for the individual patient. Care actions may be defined in three levels: the individual patient level, the plural patients' level, and the organization level.

Administrative tools may for example include all or any subset of the following:
1. Registering a patient to the system
2. Moving a patient from one clinic to another, or from one clinician to another.
3. Adding and updating information about a registered patient, such as demographics, physical, medical conditions, insurance, and type of care.
4. Discharging the patient or changing the discharge date.

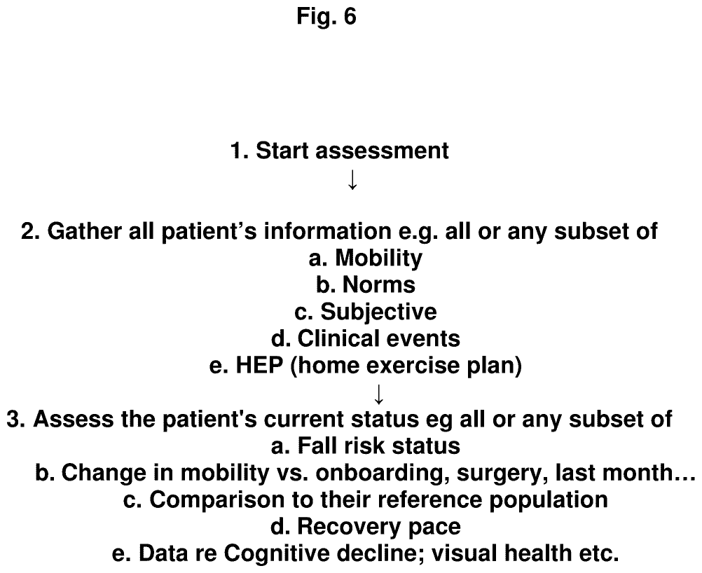

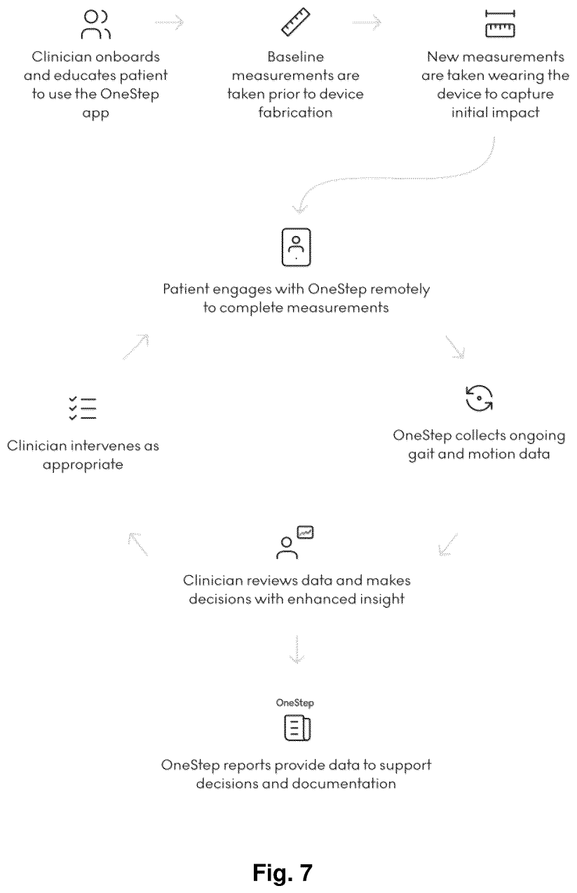

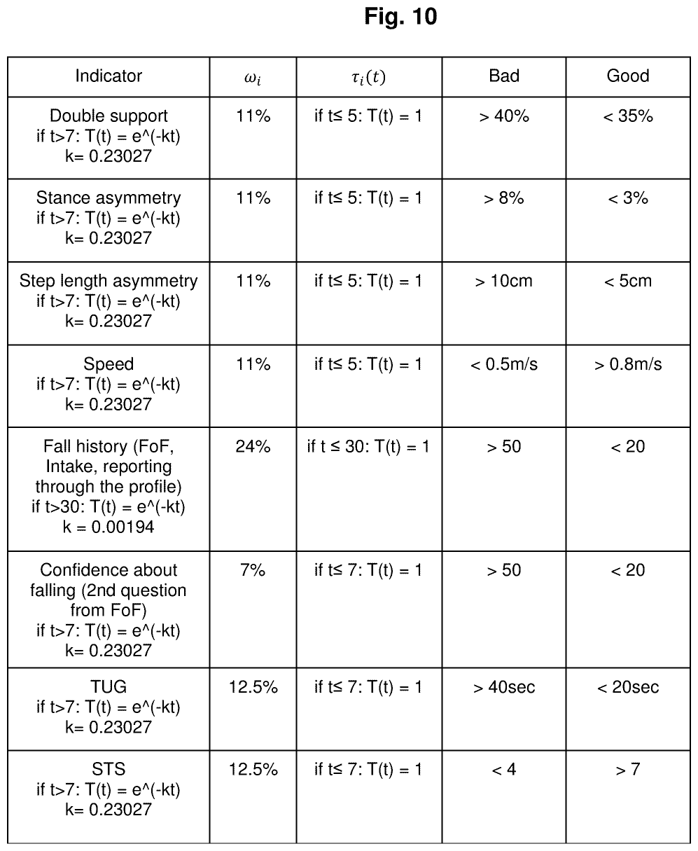

Example care actions for the individual patient are now described, which are not intended to be limiting:
1. Alerting on patients at severe risk—alert the clinician to contact the patient as soon as possible and mitigate the risk.
2. Prompting the patient to provide data—the clinician may request that the patient provide any data available to collect in the patient app, including gait analysis, standard tests, and questionnaires. This usually happens when a new patient is registered, no updated data is available, or the clinician just wants to encourage more patient engagement.
3. Justification of type of care
   a. Referrals—a recommendation supported by patient data to refer to another type of care.
   b. Justification of extension of care—a recommendation supported by patient data to extend the patient's current discharge date.
   c. Early discharge—a recommendation supported by patient data to finish the patient's treatment earlier than expected.
4. Reviewing patient's data—review of patient's indications and understand their causes (FIG. 10)
5. Strengthen documentation with objective patient data
   a. Generate progress notes and reports in seconds e.g. as shown in FIG. 3a
   b. Justify medical necessity and secure reimbursement
   c. Seamlessly integrate results into existing EMRs
6. Generate home exercise plan—Assign and adjust personalized video exercise programs
   a. Pre-built and customizable protocol templates
   b. Smart suggestions based on patient behavior and best practice
   c. Option to upload unique exercise and education videos
7. Communication with the patient (FIG. 7)—Communicate effortlessly or build a remote plan of care
   a. Get intervention suggestions based on patient data
   b. Show patients their progress and perform gait training with ‘live feedback’

Example Care actions for a group including plural patients are now described in detail; given a group of patients, there may be several options to manage care attention and choose the patients with higher needs, including but not limited to:
1. Segmentation—for example, dividing the patients into subgroups of patients with severe risk of falls, high risk of falls, and moderate and low risk levels. Segmentation may be available for any indication since every indication typically has levels of severity; and/or
2. Prioritization of patients—e.g. a sorted list of patients ordered from patients at higher risk to lower.

Care actions on the organization level which are at a higher level of care actions typically focused on evaluating treatments, clinicians, clinics and organization, may facilitate several processes, e.g.:
1. Command and control of organizational aspects: who over/underperform, in which facility patients are at higher fall risk, in which treatment patients recover faster; and/or
2. Clinical research: comparing outcome measures in different treatments.

The monitoring flow of FIG. 6 depicts a flow that may start up periodically (e.g., every day at 12 noon system time) or be triggered by a patient's activity (e.g., any communication with the service such as a mobility measurement) or by a clinician's activity (e.g., any communication with the service). Typically, the system starts the monitoring flow for the corresponding patient or patients (when it occurs periodically or is initiated by a clinician treating a group of patients). Once the monitoring flow starts for a certain patient, the service first retrieves and gathers all of that patient's data, then structures it to filter noise and outliers, and eventually assesses the patient's current status expressed as a set of indications.

Embodiments of patient's data over time, then the structuring process, including filtering and interpolation, and lastly the definition of indications and their different types that comprise the patient's status, are now described in detail.

Patient data may include information provided by the clinician during registration or updated afterward, such as demographic information, clinical conditions, and type of care. It also typically includes data provided by the patient using the patient app, including objective active and passive gait measurements, standard test analysis, and patient-reported outcomes and questionnaires. More information, such as clinical events, injuries, surgeries, and medication, may be updated throughout the treatment.

Herein, “passive” is intended to include IMU data collection from a user's phone's IMU, which does not require user cooperation each time the IMU data is collected, and in fact the user may be unaware that IMU data is being collected, e.g. because IMU data collection and subsequent analysis do not require that the user be prompted to provide any information or to perform any particular action. For example, the system typically does not require a user to be prompted to walk or to indicate whether or not s/he is walking. Instead, the disclosures of co-owned U.S. Ser. No. 18/975,890 entitled “Advanced Pedestrian Navigation Based on Inertial Gait Analysis and GPS Data” and/or of co-owned US20220111257 entitled “System . . . for Sensor-Based Enhancement of Physical Rehabilitation” (the disclosures of which are both hereby incorporated herein by reference in their entirety) may be used to automatically classify a given time-interval as including gait or not e.g. by determining whether the user is ambulating during that interval. Then, IMU data may, if desired, be collected only during such intervals which, as automatically determined, do include ambulation, as opposed to intervals in which an end-user is stationary, for example.

Any suitable structure of a patient's longitudinal representation may be employed. Typically, eventually, all patient data is structured on the patient's timeline, allowing assessment of various indications, which depend on the patient's conditions and the type of care. Before applying the indications' assessment process, the patient's timeline may be processed by all or any subset of the following procedures, suitably ordered e.g. as follows:
1. Outlier filtering—sometimes there may be some discrepancies in the estimated mobility measures. This is even more likely in passive gait measurements. In order to reduce such noisy data, the system may be configured to filter out (or at least indicate with lower certainty) strange data points. This task is possible when the patient's timeline is available and enables the comparison of data points and the patient's reference. For example, a gait measurement with a gait speed higher or lower by three standard deviations from the patient's weekly average gait speed may be considered an outlier. When many data points are available, as in passive gait monitoring, a daily or weekly (or any other timespan) aggregation, taking the median or the average, may be another solution to reduce outliers' noise.
2. Interpolation of trends—Mobility measures (such as gait speed, stance asymmetry, ranges of motion, and PROs) usually have a continuous characteristic behavior in time, unless a dramatic event occurs (such as an injury). For this reason, it is reasonable to interpolate some mobility measures between data points, as long as no medical incident happens, and produce trends for those measures. A standard way for interpolation may be linear interpolation between data points, and using a sliding window that averages the interpolated data to reduce measurement fluctuations afterward. The more frequent the measurements are, the more representative the trend line is. Gait active measurements may be taken between a few times a week and once every two weeks, depending on the natural progression of the patient's condition. The sliding window length may be determined accordingly, to match the measurement frequency the patient is required in the program.
3. Modifying norms and benchmarks—norms are aggregations of patients' data and timelines over a specific population, for example, gait parameter norms by demographic information, such as normative values of gait speed by age and gender. Another example is the recovery pace in different parameters or outcome measures post-surgery, for instance, benchmarks of 6-minute walk test distance by week for different surgeries and populations. Norms and benchmarks may be hardcoded supported by literature, or computed by observations. Hence, when a modified timeline is obtained, there is an opportunity to regenerate norms. This process may alternatively run after a large set of new timelines and patient data is collected. Norms may become as granular and specific as available, e.g., gait norms for patients with Parkinson's disease, aka PD, by gender, age, height, and weight.

An “indication” as used herein typically represents all or any subset of a level of severity, functional assessment, progression of recovery, or deterioration in any medical and health-related condition at a specific point on the patient's timeline. For example, a fall risk indication of severe risk of fall indicates that a patient is assessed with a severe risk of falling on a specific date or period of time. Thus, an indication is a function of a patient's timeline at a specified time.

An indication may compare the patient's timeline and data to the patient's relevant norms, e.g., an indication of how fast or slow this patient recovers from a total hip replacement compared to the recovery benchmark of a population of the same age and gender post-total hip replacement surgery.

Each indication has a level, e.g., positive, neutral, and negative, indicating whether it is a good or bad indication. It also enables comparing it to another indication of the same patient the same kind of indication but at another time specified, or another patient's indication of the same kind, and even between different kinds of indications. This allows for the sorting and segmenting of patients by indications. One way to implement this is to provide an indication with a numerical score, which may be presented in the clinician dashboard and the patient app.

Each indication is explainable using the formula that yields it and the values of the contributing measures included in the patient's data, the timeline's properties (such as mobility measure trends), norms, and benchmarks. An indication may be nested with other indications. For example, a cognitive decline indication may be defined by comparing two consecutive cognitive impairment indications. An explanation of a nested indication is recursive.

Indications may include any of the following: fall risk, cognitive impairment, post-op recovery, progression of Parkinson's Disease, mobility affected by medication, adaptation to assistive devices or prosthetics, visual impairment, diabetic foot status, back chronic pain, spine pain mobility status, functional status of patients treated with chemotherapy, and clinical depression. A possible operationalization of a fall risk indication is described:

An example fall risk indication, which assesses the patient's risk of falls, may be computed as follows:

$$score ( t ) = \frac{{\sum }_{i} ( {τ}_{i} ( t ) {ω}_{i} \cdot {s}_{i} )}{Ω ( t )}$$

$$Ω ( t ) = {\sum }_{i} {τ}_{i} ( t ) {ω}_{i}$$

where:
i—a contributor component or gait parameter such as, for example:
   Stance asymmetry (Walk)
   Step length asymmetry (Walk)
   Double support (Walk)
   Velocity (Walk)
   FoF score (FoF questionnaire)
   TUG duration (TUG)
   STS repetitions (STS)
sᵢ—specific score attributed to that component in categorial terms—bad, neutral, and good (−1,0,1). Neutral is when the component's value does not match the bad or good criteria.
τᵢ(t)—part of the weight attributed to the component based on the time it was measured. e.g. how close a given measurement is to expiration
ωᵢ—optional weight attributed to each component based on its clinical meaning and/or importance and/or relevance.

However, any suitable method may be used to generate a level (e.g. high/mid/low) of fall risk. For example, fall risk may be computed as a sum of factors, each factor being a combination, e.g. a multiplicative product of:
a score (e.g. score/s for some or all questions in FoF-fear of falls questionnaire, TUG—time up and go score, STS—sit to stand score) or gait parameter (e.g. an asymmetry parameter e.g. stance asymmetry, step length asymmetry, or double support or velocity) or other typically ordinal factor e.g. fall history; and/or
a recency weight assigned to the score or gait parameter (high if recent, low otherwise); and/or
a clinical importance weighting parameter e.g. if all of the above scores and parameters are used (thus the fall risk is computed as a sum of plural factors) then all may be weighted as equally clinically important other than fall history which might be weighted higher, whereas confidence about falling (2^{nd} question from FoF) might be weighted lower.

The table of FIG. 10 defines example parameter values for each component.

The total score of the fall risk indication may, for example, be considered good if the score is greater than 0.2, bad if it is lower than −0.2, and neutral otherwise.

Any suitable care action rules and customization may be employed. Typically, when a patient's indications are computed, they allow different care actions to be defined, depending on their values by a rule-based mechanism. This mechanism may be specified on the system setup, or modified and customized later.

Examples of justification of care are now described. Justification of a care action may itself be defined as a care action. The system is typically configured to document why (e.g. on the basis of which indications) the system has recommended a certain care action.

Referrals: There are several ways a patient may be referred back to a new episode of care, e.g. all or any subset of the following:
1. Transition from non-skilled to skilled therapy within a facility. This may be necessary when a resident has deteriorated or has become a high fall risk.
2. Transition from in patient skilled to outpatient with RTM. When patients leave an acute or sub-acute facility which is covered under Med A, they may be referred to an outpatient or RTM program under Med B.
3. Remote monitoring of resident in an ALF aka assisted living facility or a former patient living independently who shows a decline in status. Noting the decline early and intervening proactively by referring to skilled care not only may prevent an adverse medical event, it enhances care and keeps the patient in a care continuum which may reduce downstream costs.
4. In order to accurately identify a referral, all patients registered must according to certain embodiments be classified as ‘non-skilled’ or ‘skilled’ and have current data.

In the event that extension of a care plan is effected for a given patient, the system may provide the evidence a carer may need to extend the plan of care for your patient to ensure they have a safe transition home to optimize their level of independence. The system may alert the carer in the following conditions:
1. Fall risk is high near the discharge date thus identifying an opportunity to extend care.
2. When the patient has deteriorated and needs more care, an adjustment to the care plan or needs continued monitoring

Early Discharge: The system may identify and provide the objective data for patients who are advancing in their recovery quickly and may be discharged home earlier than anticipated.

Any suitable technique/s may be employed to achieve customization of care actions, such as but not limited to all or any subset of the following:
1. Trigger—The conditions that trigger the care actions may be adjusted according to specific customer preferences. For example, customers who measure their patients daily may be informed of significant changes (decline or improvement in motion) based on daily data, while those who measure on a weekly basis may be notified only of weekly changes.
2. Text—The text of the care action, including the description, explanation, and call to action, may be customized based on the customer's specific content and style preferences. Care actions may be modified to be more descriptive or prescriptive in style.
3. Presentation Time—The duration a care action appears before it disappears. For example, some customers may prefer having a care action such as “Missing mobile permissions” remain visible until resolved, while others may prefer it to appear for only three days after being triggered.
4. Cooldown Time—The interval between appearances of the same care action after it has been triggered. For example, some customers may prefer a 2-week interval between “patient is declining in motion” notifications, while others may prefer only a 3-day interval.
5. Medium—Care actions are configured to appear in the clinician web dashboard, the clinician app, as a mobile push notification, and as part of a periodic email. This configuration is set per action.
6. Packages—Care actions are grouped into packages representing different themes in the continuum of care. To ease the decision-making process, customers may choose to opt into specific packages instead of browsing care actions one by one.

An all Patients View may be provided, which may comprise a page in the clinician view where all of the clinician's patients are presented, and enables triaging (plural patient care actions), and immediate individual patient care actions, such as alerting on patients at a high risk.

A clinician may seek to add/remove columns to obtain get a bird's eye view on the fields that matter to his/her day-to-day work. Typically, each column may also be accompanied by its own unique icon and/or, when adding columns that exceed the width of the screen a horizontal scroll may be introduced. The first column (patient name) is: a) mandatory, cannot be removed and b) fixed. This means that when scrolling right, it may persist in its location </aside>.

A clinician may seek to sort the all patients table by any displayed column so that the right patient in the right context that is of interest at that particular moment, may be seen.

Prioritize Patients: a clinician may seek to visually identify which patients need attention at a specific moment, enabling prioritization of time spent. Priorities explained: (example)
1. SNFs: We may start with coupling the field to fall risk
2. RTM: Each patient has max. 3 care action items assigned, each has a priority score.
   1. Top priority are all patients that have any care action item with a priority score=0. Within those, compute average priority score for each patient, sort by average (lower is top). Mark all with red color.
   2. Repeat the same process for priority score=1, then 2, etc. Mark all with a neutral color (not yellow or green); the Priority-0 patients show some sort of clinical urgency due to decline, hence say red color. For other action items, the prioritization is not on the negative-positive spectrum, but on a time sensitivity spectrum, therefore yellow and green colors may be misleading. A neutral color that communicates “actionability” is preferable.

Any suitable measurement protocol field may be employed; FIG. 11 is provided in this connection merely by way of example.

A clinician may seek to understand the measurement protocol status at a glance to obtain immediate orientation on where he/she should focus active efforts at that time. Protocol explanation: example:
1. STS+TUG→
   1. 1 STS and 1 TUG needed once every two weeks.
   2. Indicator details:
      1. 100% (full circle)→if measurement was taken in the last 7 days
      2. 50% (half circle)→if measurement was taken between 7 and 14 days
      3. 0% (empty circle)→if measurement was taken 14+ days ago
2. Walk→
   1. 2 walks a week are needed
   2. Indicator details: (2 walks indicators)
      1. 100% (full circle)→if walk was taken in the last 5 days
      2. 50% (half circle)→if measurement was taken between 5 and 7 days
      3. 0% (empty circle)→if measurement was taken 7+ days ago
3. FOF (Fear of falls)
   1. 1 FoF questionnaire every 30 days is needed
   2. Indicator details:
      1. 100% (full circle)→if questionnaire was answered less than 15 days ago
      2. 50%—between 15-30 days ago
      3. 0% (empty circle)→if questionnaire was answered 30+ days ago
4. Choosing which measurements to display:
   1. Always take the most recent measurements. In case of walks—last two measurements
5. Hover state:
   1. All hover states may include all of any subset of:
      1. Is it up-to-date or expired?
         1. If expired—“This measurement was expired 3 days ago”
         2. If up-to-date—“Up-to-date. This measurement will expire in 3 days”
         1. If 1 day ago—“yesterday”
         2. in 1 days—“tomorrow”
         3. if today—“today”
      2. Last measurement score
      3. Last measurement date
      4. Educational info:
         1. STS: “STS is required once every 14 days”
         2. TUG: “TUG is required once every 14 days”
         3. Walk: “Walk measurement is required twice a week”
2. Hover States may be provided.

A Timed up and Go (TUG) screen display may indicate, say:
Last measurement: Mar. 12, 2023
Score: 18 sec
Expiration: expires in 4 days
Protocol requirement: Perform once every 14 days

The term ‘episode of care’ is used herein when referring to a system ability to define the exact dates a patient is receiving a specific type of care. For example, a patient may be receiving physical, occupational, and speech therapy for ‘skilled care,’ or they may be participating in a restorative program or ‘non-skilled’ care.

Restorative care or RNA plays an important role in a PDPM (patient-driven payment model). Nursing services are reimbursed for this model if they have documentation of restorative interventions. When the system herein is deployed in a robust RNA program, all the data required may be captured so that the facility may receive reimbursement.

F tags (federal tags) are used by CMS (center for Medicare Services) to identify areas of deficiencies. When residents participate in a restorative walking and mobility program and it is documented, F tags may be avoided.

It is best practice to designate which patients are in a skilled or non-skilled model of care. There are at least the following ways, for example, to make this distinction in a patient population:
1. Choose care type on patient intake
2. Choose or change the care type under profile—episode of care tab in the clinic portal.
   When changing care types, the system may require to first start a new episode of care.
   Go to profile—episode of care—start new episode
      choose an anticipated discharge date. This date is for informational purposes only and may not result in any outcomes if the date is passed and the patient is still active
   Choose the type of care.
   Add an anticipated discharge date. This may help identify patients who remain at a high fall risk as they approach their discharge date, and it may be an opportunity for extension of care.
   the carer typically cannot change the episode of care more than once a day.
3. Patients who are classified as non-skilled and show a decline in function may be flagged by the care engine to alert the attending provider to assess. This may be an opportunity to change episode of care to ‘skilled’ and count as a new referral to therapy.

### Opportunities after Discharge from Skilled Services

Many skilled care organizations offer a full continuum of care for the aging adult. There may be a sub-acute skilled nursing facility (SNF) with rehabilitation on the same campus as an assisted living (AL) center or an independent living center (IL).

In this continuum, the patient may reside in the AL or IL, experience a medical event, be hospitalized, and then transferred to the skilled nursing center for further care until they are well enough to return to their previous living environment. That previous environment may be the AL or IL of the organization, or some patients may reside in the community and their goal is to return home.

In either case, the skilled nursing facility aka SNF may offer continued therapy on an outpatient basis to ensure the patient continues to make progress toward functional goals and optimize independence, or the patient may qualify for skilled maintenance. Either way, the clinician may easily continue the care through the OneStep system, regardless of the final destination of the patient. Once the patient is utilizing outpatient services through Medicare, therapy charges may go through part B and thus qualify for Remote therapeutic monitoring aka RTM. The OneStep system makes it easy to invite the patient to download the OneStep app on their phone so they may record walks on their own. To invite the patient, tap on patient card in the clinician app then look for the three dots in the upper right-hand corner. Tap on those dots and there may be a pop up at the bottom of the screen to share the link to the patient. Tap on the link to see a field to add the patient's phone number and the following options:
QR code
Send SMS—this may be the default option
Patient verification—Once the patient receives the SMS or scans the QR code, they may then be asked to verify the phone by inserting a 4-digit code they receive by SMS from the OneStep system. In this way, the phone number associated with the clinician app may not be shared with the patient.

Once this is done, if the patient logs out of the app, they may always easily sign back into their account.

The following describes how the system herein may enhance restorative care:

Joe is a 75-year old male resident in SNF. He participates in a restorative nursing program which entails a 15 min daily walk with an RNA aka restorative nurse assistant. The restorative nurse assistant aka RNA may attach a commercially available OneStep strap to Joe e.g. if he has no pocket and record a walk with the OneStep app 2× a week. Once a week, the Director of Rehab (DoR) may generate a progress note in the clinic portal and review the gait data of all the residents. The DoR notices Joe has a decline in several gait parameters. His speed decreased by 0.2 m/sec and his double support increased to 45%. She also noticed that Joe has increased step length asymmetry. She orders a physical therapy evaluation. Upon assessment, the therapist notes Joe's dynamic balance is poor and that he has complained of pain in his right leg. Joe is brought back onto caseload for skilled therapy to address these impairments. The therapist may need to establish a new episode of care for skilled therapy in the profile-episode of care section where an “episode” of care refers to a period in which a given patient is treated using a specific type of care. Typically, this referral may fall under Medicare part B, since Joe is a long-term resident. The referral is an economic opportunity for the facility for a new source of revenue from part B, and, most importantly, they may have mitigated a potential adverse medical event, such as a fall.

It is appreciated that to achieve a reduction in falls, all or any subset of the following best practices for utilizing fall prevention and care enhancement for inpatient facilities may be provided.

A strap may be used to secure a phone to, say, the front thigh of the patient, and allows the clinician to see the phone and toggle through the screens without removing it from the strap.

The system herein may be used daily to record a walk or test during evaluations, Recording walks may be a part of gait training.
Recording TUG may be used in gait training or therapeutic activity.
Recording STS may be used during therapeutic exercise or therapeutic activity.

The system herein may be used to create comprehensive and customizable weekly progress reports. The objective data collected may be used to inform clinical decision-making, and justify or extend care.

Therapy aides may be helpful in facilitating operation of the system herein; they may register patients and have the phones ready to go when needed. RNAs may record walks with the patients on their walking programs. An organization may assign a therapist to analyze the data of patients on an RNA program for 15 min weekly to identify patients in need of skilled care. When RNAs use the system herein, the organization or facility may preventively identify potential fallers, and decrease F-flags for the facility.

According to an embodiment, one side of the patient card on the ‘All Patient’ view page may turn green, yellow, or red, based on the priority of tasks needed for optimal patient care. For example, for visualization, red means high priority, yellow means medium priority, and green means low priority.

The system herein may trigger a care action to capitalize on an opportunity to extend care, and/or highlight a patient that is declining, or remains at a high fall risk near discharge.

Care actions may be configured and individualized to each organization's needs. For example, a patient who declines, and moves from a medium to high fall risk, may be prioritized over a patient who has consistently been at a high fall risk. Or, a patient who is flagged as SBA (oris about to be discharged by an organization) but is a high fall risk, may be prioritized as an opportunity to extend his treatment and provide better care.

According to embodiments herein, even non-ambulatory patients may be tested. The Functional reach test, and FoF and STS for patients who are unable to walk and obtain meaningful insights, may be used.

It is appreciated that the system may control allocation of referrals of end-users of an organization Oj, by the organization Oj (which is an organizational user of the system, hence has rules governing such referrals), to an organization Ok which is external to the system, is not an organizational end-user of the system, and does not itself have the interface and the processor.

It is appreciated that a biomarker or indication may be trained that estimates indexes directly from IMU dataas opposed to, or in addition to, using functions of mobility measures to derive or compute or estimate or predict indications.

Thus for example, a trained model may be applied directly to imu data in order to directly predict or estimate presence of shuffling gait.

An advantage of certain embodiments is that the professionalism of modern medicine can paradoxically be the very factor which impedes its further progress. Specifically, despite a large body of knowledge accumulating in the field of gait analysis, gait analysis outputs are insufficiently used, to date, if at all, to systematically guide allocation of finite healthcare organization resources. One reason is that gait parameters are inferior to known “standard outcomes” (which may be clinical/functional/quality of life measures including results of tools or scores administered/assigned in the clinic, PROs etc), in terms of ease of interpretation. Because clinicians are, by definition, unused to relying on gait analysis outputs, these outputs are, at this time, harder for health professionals to interpret.

Embodiments herein overcome these problems e.g. by providing a system which almost costlessly (no hardware, reliance on cellphones which patients use anyway) estimates whatever criteria (clinical test results, PROs, etc.) are currently used in health providing organizations to enhance ease of interpretation of an individual's gait analysis parameters. For example if TUG scores are conventionally used to determine whether or not a gait aid should be allocated to a given end-user, these scores can be estimated from the imu data generated by the cellphones of end-users of the system according to embodiments herein. Or, if patients with A1c≥6.5% are assigned to, say, metformin or insulin, then Alc and/or FPG and/or OGTT scores can be estimated from the imu data generated by the cellphones of end-users of the system according to embodiments herein. Or, if “the 6MWT is used as a clinical basis for prescribing oxygen for home use” (pmc.ncbi.nlm.nih.gov/articles/PMC9095083/), 6 minute walk test scores can be estimated from the imu data generated by the cellphones of end-users of the system according to embodiments herein, and similarly for any cancer (say) markers or tests used to determine resource allocation to and between cancer (or other diseased) patients.

Then, e.g. if treatments and their outcomes are fed back into the system, the system continues to learn to enable even better recommendations to be generated in future, e.g. as the system matures, by more direct reliance on gait parameters and fluctuation thereof over time.

Inter alia, the respective confidence levels of system predictions of various standard outcomes or tests can be computed to enable clinicians to evaluate whether the system herein can currently be relied upon to drive resource allocation decisions which rely upon these specific outcomes or tests and of course, as the system matures, these confidence levels may rise, enabling clinicians to adopt system recommendations selectably (to predict certain standard outcomes or tests but not others) in an evidence-based manner.

Yet another advantage of embodiments herein, is related to the fact that blood (or urine) tests are known, in modern evidence-based medicine including the science of health administration, to be crucial far beyond their role in diagnosing actual diseases of the blood like leukemia. The versatility of blood tests may allow not only blood tests but also gait analysis to serve as powerful tools, separately or together, for resource allocation in the course of managing a wide variety of diseases, in evidence-based health administration. Yet advancement toward that future can only occur if a system is proposed which: (a) allows the potential of gait analysis in resource allocation to and/or between, say, cancer patients to be learned; and/or (b) does not require risks to be taken (e.g. by abandoning allocation practices generated the old way or by re-routing resources to expensive data collection, rather than these resources being allocated to other admittedly useful purposes as they are today) and/or (c) does not require burdensome data collection (burdensome for clinicians and/or for patients and/or in terms of cost borne by organizations), since currently, the advisability of resource allocation in reliance on gait analysis e.g. as described herein has yet to be proven scientifically. Embodiments herein achieve this.

It is appreciated that the advantages of this system as opposed to conventional healthcare system may include continuous screening for or identification of entirely new medical issues e.g. new infection, new stroke, new fracture/fall. For example, if on Tuesday morning, Grandma Julia is suddenly walking much more slowly, or is limping, or is walking asymmetrically, or is not walking at all, the organization may get or generate an alert, and alert, prioritize, and route to org's resources and solutions and services, according to output/s of the continuous screening, according to rules.

It is appreciated that any suitable output indication generating functionality may be employed to generate an output indication of any result or prediction or parameter described herein, which may be yielded by any embodiment of the system herein; the output indication may be provided to any suitable entity in any suitable manner (e.g. automatic SMS to HMO and/or a report document which may be consumed via web dashboard or via API with EMR software platform such as Epic Systems or Cerner, or via email to an email address, stored in the system, of the entity).

Any operation described herein as being “continuous” may alternatively simply be automatically performed repeatedly, e.g. according to a programmed schedule, or periodically.

According to certain embodiments, the system provides an accurate indication of which end-users of each organization most require organizational resources/services at a given time e.g. today.

According to certain embodiments, the system may be configured to learn over time which types of accelerometer readings are indicative of the most urgent emergencies e.g. heart attacks and strokes, for example if labelled data is available indicating deaths of certain end-users, IMU readings prior to the death event may be gait-analyzed to identify patterns of imu readings which are typical of these deaths.

Certain embodiments provide an ongoing online learning process which learns the relationship between indications and whatever care actions (interventions—typically including first aid as one type of care action) were executed and recommends care actions accordingly for various users with various indications, as the system learns.

It is appreciated that many use-cases exist which may benefit from continuous measurement of cognitive status using a phone, such as but not limited to organizations seeking to assign interventions. Other use-cases which may benefit from continuous measurement of cognitive status using a phone, include computerized systems (e.g. serving pharma, med-device companies, research academic institutions) which are tasked with managing clinical trials of the safety of drugs or treatment efficacy and which may use the system herein for monitoring the mobility of patients with neurological issues.

It is appreciated that terminology such as “mandatory”, “required”, “need” and “must” refer to implementation choices made within the context of a particular implementation or application described herewithin for clarity, and are not intended to be limiting, since, in an alternative implementation, the same elements may be defined as not mandatory and not required. or may even be eliminated altogether.

Components described herein as software may, alternatively, be implemented wholly or partly in hardware and/or firmware, if desired, using conventional techniques, and vice-versa. Each module or component or processor may be centralized in a single physical location or physical device or distributed over several physical locations or physical devices.

Included in the scope of the present disclosure, inter alia, are electromagnetic signals in accordance with the description herein. These may carry computer-readable instructions for performing any or all of the operations of any of the methods shown and described herein, in any suitable order, including simultaneous performance of suitable groups of operations, as appropriate. Included in the scope of the present disclosure, inter alia, are machine-readable instructions for performing any or all of the operations of any of the methods shown and described herein, in any suitable order; program storage devices readable by machine, tangibly embodying a program of instructions executable by the machine to perform any or all of the operations of any of the methods shown and described herein, in any suitable order, i.e., not necessarily as shown, including performing various operations in parallel or concurrently, rather than sequentially, as shown; a computer program product comprising a computer useable medium having computer readable program code, such as executable code, having embodied therein, and/or including computer readable program code for performing, any or all of the operations of any of the methods shown and described herein, in any suitable order; any technical effects brought about by any or all of the operations of any of the methods shown and described herein, when performed in any suitable order; any suitable apparatus or device or combination of such, programmed to perform, alone or in combination, any or all of the operations of any of the methods shown and described herein, in any suitable order; electronic devices each including at least one processor and/or cooperating input device and/or output device and operative to perform, e.g., in software, any operations shown and described herein; information storage devices or physical records, such as disks or hard drives, causing at least one computer or other device to be configured so as to carry out any or all of the operations of any of the methods shown and described herein, in any suitable order; at least one program pre-stored e.g. in memory or on an information network such as the Internet, before or after being downloaded, which embodies any or all of the operations of any of the methods shown and described herein, in any suitable order, and the method of uploading or downloading such, and a system including server/s and/or client/s for using such; at least one processor configured to perform any combination of the described operations or to execute any combination of the described modules; and hardware which performs any or all of the operations of any of the methods shown and described herein, in any suitable order, either alone or in conjunction with software. Any computer-readable or machine-readable media described herein is intended to include non-transitory computer- or machine-readable media.

Any computations or other forms of analysis described herein may be performed by a suitable computerized method. Any operation or functionality described herein may be wholly or partially computer-implemented, e.g., by one or more processors. The invention shown and described herein may include (a) using a computerized method to identify a solution to any of the problems or for any of the objectives described herein, the solution optionally including at least one of a decision, an action, a product, a service or any other information described herein that impacts, in a positive manner, a problem or objectives described herein; and (b) outputting the solution.

The system may, if desired, be implemented as a network—e.g., web-based system employing software, computers, routers, and telecommunications equipment, as appropriate.

Any suitable deployment may be employed to provide functionalities, e.g., software functionalities shown and described herein. For example, a server may store certain applications, for download to clients, which are executed at the client side, the server side serving only as a storehouse. Any or all functionalities, e.g., software functionalities shown and described herein, may be deployed in a cloud environment. Clients, e.g., mobile communication devices such as smartphones, may be operatively associated with, but external to the cloud.

The scope of the present invention is not limited to structures and functions specifically described herein and is also intended to include devices which have the capacity to yield a structure, or perform a function, described herein, such that even though users of the device may not use the capacity, they are, if they so desire, able to modify the device to obtain the structure or function.

Any “if-then” logic described herein is intended to include embodiments in which a processor is programmed to repeatedly determine whether condition x, which is sometimes true and sometimes false, is currently true or false, and to perform y each time x is determined to be true, thereby to yield a processor which performs y at least once, typically on an “if and only if” basis, e.g., triggered only by determinations that x is true, and never by determinations that x is false.

Any determination of a state or condition described herein, and/or other data generated herein, may be harnessed for any suitable technical effect. For example, the determination may be transmitted or fed to any suitable hardware, firmware, or software module, which is known or which is described herein to have capabilities to perform a technical operation responsive to the state or condition. The technical operation may, for example, comprise changing the state or condition, or may more generally cause any outcome which is technically advantageous, given the state or condition or data, and/or may prevent at least one outcome which is disadvantageous, given the state or condition or data. Alternatively or in addition, an alert may be provided to an appropriate human operator or to an appropriate external system.

Features of the present invention, including operations which are described in the context of separate embodiments, may also be provided in combination in a single embodiment. For example, a system embodiment is intended to include a corresponding process embodiment, and vice versa. Also, each system embodiment is intended to include a server-centered “view” or client centered “view”, or “view” from any other node of the system, of the entire functionality of the system, computer-readable medium, apparatus, including only those functionalities performed at that server or client or node. Features may also be combined with features known in the art, and particularly, although not limited to those described in the Background section or in publications mentioned therein.

Conversely, features of the invention, including operations, which are described for brevity in the context of a single embodiment or in a certain order, may be provided separately or in any suitable sub-combination, including with features known in the art (particularly although not limited to those described in the Background section or in publications mentioned therein) or in a different order. “e.g.” is used herein in the sense of a specific example which is not intended to be limiting. Each method may comprise all or any subset of the operations illustrated or described, suitably ordered e.g. as illustrated or described herein.

Devices, apparatus or systems shown coupled in any of the drawings may in fact be integrated into a single platform in certain embodiments, or may be coupled via any appropriate wired or wireless coupling, such as but not limited to optical fiber, Ethernet, Wireless LAN, HomePNA, power line communication, cell phone, Smart Phone (e.g. iPhone), Tablet, Laptop, PDA, Blackberry GPRS, Satellite including GPS, or other mobile delivery. It is appreciated that in the description and drawings shown and described herein, functionalities described or illustrated as systems and sub-units thereof can also be provided as methods and operations therewithin, and functionalities described or illustrated as methods and operations therewithin can also be provided as systems and sub-units thereof. The scale used to illustrate various elements in the drawings is merely exemplary and/or appropriate for clarity of presentation, and is not intended to be limiting.

Any suitable communication may be employed between separate units herein, e.g., wired data communication and/or in short-range radio communication with sensors such as cameras e.g., via WiFi, Bluetooth, or Zigbee.

It is appreciated that implementation via a cellular app as described herein is but an example, and, instead, embodiments of the present invention may be implemented, say, as a smartphone SDK; as a hardware component; as an STK application, or as suitable combinations of any of the above.

Any processing functionality illustrated (or described herein) may be executed by any device having a processor, such as but not limited to a mobile telephone, set-top-box, TV, remote desktop computer, game console, tablet, mobile e.g. laptop or other computer terminal, embedded remote unit, which may either be networked itself (may itself be a node in a conventional communication network e.g.) or may be conventionally tethered to a networked device (to a device which is a node in a conventional communication network, or is tethered directly or indirectly/ultimately to such a node).

Any operation or characteristic described herein may be performed by another actor outside the scope of the patent application and the description is intended to include apparatus whether hardware, firmware or software which is configured to perform, enable, or facilitate that operation or to enable, facilitate, or provide that characteristic.

The terms processor or controller or module or logic as used herein are intended to include hardware such as computer microprocessors or hardware processors, which typically have digital memory and processing capacity, such as those available from, say Intel and Advanced Micro Devices (AMD). Any operation or functionality or computation or logic described herein may be implemented entirely or in any part on any suitable circuitry including any such computer microprocessor/s as well as in firmware or in hardware or any combination thereof.

It is appreciated that elements illustrated in more than one drawing, and/or elements in the written description, may still be combined into a single embodiment, except if otherwise specifically clarified herewithin. Any of the systems shown and described herein may be used to implement or may be combined with, any of the operations or methods shown and described herein.

It is appreciated that any features, properties, logic, modules, blocks, operations, or functionalities described herein which are, for clarity, described in the context of separate embodiments, may also be provided in combination in a single embodiment, except where the specification or general knowledge specifically indicates that certain teachings are mutually contradictory and cannot be combined. Any of the systems shown and described herein may be used to implement or may be combined with, any of the operations or methods shown and described herein.

Conversely, any modules, blocks, operations or functionalities described herein, which are, for brevity, described in the context of a single embodiment, may also be provided separately or in any suitable sub-combination, including with features known in the art. Each element, e.g., operation described herein may have all characteristics and attributes described or illustrated herein, or, according to other embodiments, may have any subset of the characteristics or attributes described herein.

It is appreciated that apps implementing any functionality herein may include a cell app, mobile app, computer app, or any other application software. Any application may be bundled with a computer and its system software, or published separately. The term “phone” and similar used herein is not intended to be limiting and may be replaced or augmented by any device having a processor, such as but not limited to a mobile telephone, or also set-top-box, TV, remote desktop computer, game console, tablet, mobile, e.g., laptop or other computer terminal, embedded remote unit, which may either be networked itself (may itself be a node in a conventional communication network e.g.) or may be conventionally tethered to a networked device (to a device which is a node in a conventional communication network or is tethered directly or indirectly/ultimately to such a node). Thus, the computing device may even be disconnected from e.g., WiFi, Bluetooth, etc., but may be tethered directly or ultimately to a networked device.

References herein to “said (or the) element x” having certain (e.g., functional or relational) limitations/characteristics, are not intended to imply that a single instance of element x is necessarily characterized by all the limitations/characteristics. Instead, “said (or the) element x” having certain (e.g. functional or relational) limitations/characteristics is intended to include both (a) an embodiment in which a single instance of element x is characterized by all of the limitations/characteristics and (b) embodiments in which plural instances of element x are provided, and each of the limitations/characteristics is satisfied by at least one instance of element x, but no single instance of element x satisfies all limitations/characteristics. For example, each time L limitations/characteristics are ascribed to “said” or “the” element X in the specification or claims (e.g. to “said processor” or “the processor”), this is intended to include an embodiment in which L instances of element X are provided, which respectively satisfy the L limitations/characteristics, each of the L instances of element X satisfying an individual one of the L limitations/characteristics. The plural instances of element x need not be identical. For example, if element x is a hardware processor, there may be different instances of x, each programmed for different functions and/or having different hardware configurations (e.g., there may be 3 instances of x: two Intel processors of different models, and one AMD processor).

1. A computerized healthcare system providing healthcare remotely to end-users, the system comprising at least one instance of:
an interface uploading IMU data recorded by a healthcare-providing organization O's end users' communication devices' accelerometers; and
a hardware processor which maps indications derived from the IMU data to care actions, where said care actions comprise at least one allocation of at least one organizational resource from among a set of available organizational resources which the organization allocates to its end-users or patients or members,
thereby to reliably and efficiently allocate available organizational resources to at least one organization's patients.

2. The system of claim 1 wherein the processor is configured to predict externally generated scores from raw data recorded by the accelerometers.

3. The system of claim 1 wherein the processor comprises a biomarker or indication that estimates indexes directly from IMU data recorded by the accelerometers.

4. The system of claim 2 wherein the processor comprises at least one classifier trained to predict the externally generated scores from the raw data recorded by the accelerometers.

5. The system of claim 4 wherein the externally generated scores comprise at least one score generated by at least one human clinician and the classifier is trained using, for training data, scores generated by at least one human clinician for at least one individual, paired with raw data recorded from the at least one individual by the accelerometers.

6. The system of claim 4 wherein the externally generated scores comprise at least one score derived from a patient questionnaire, and the classifier is trained using, for training data, scores derived from a patient questionnaire filled out by at least one individual, paired with raw data recorded from the at least one individual by the accelerometers.

7. The system of claim 2 wherein the externally generated scores comprise scores indicative of a motor disease such as but not limited to, multiple sclerosis or Parkinson's or malfunction of lower extremities.

8. The system of claim 2 wherein the externally generated scores comprise at least one of:
UPDRS scores
PDQ scores
EDSS scores generated by a human clinician such as a neurologist
PDDS scores derived from a PDDS patient questionnaire
LEFS scores derived from an LEFS patient questionnaire
EQ-5D scores
SF-36 or SF-12 scores.

9. A system according to claim 1 which includes plural instances of the interface (e.g. plural secure data channels) and/or of the processor, for plural healthcare providing organizations O1, . . . ON respectively, and wherein said organizational resources, which at least one organization Oj allocates to its end-users or patients or members, includes referrals to outside organizations, other than organization Oj, which typically perform care actions not performed within the organization Oj.

10. The system of claim 1 wherein recording of IMU data is unobtrusive and/or frequent and/or continuous and/or does not require activation and/or passive.

11. The system of claim 1 wherein said indications comprise characterizations of extent of motion during time-interval/s, to identify time-windows in which an end-user was immobile, or less mobile, and which are unexpectedly or uncharacteristically long.

12. The system of claim 2 wherein the externally generated scores comprise scores indicative of a respiratory disease such as but not limited to asthma or chronic obstructive pulmonary disease (COPD).

13. The system of claim 1 wherein at least one indication from among the indications derived from patient P's IMU data comprises a putative new diagnosis for patient P, such as a possible new stroke or possible new fall or possible new bout of influenza or possible new fracture.

14. A computerized healthcare method providing healthcare remotely to end-users, the method comprising providing at least one instance of an interface uploading IMU data recorded by a healthcare providing organization O's end users' communication devices' accelerometers; and
providing at least one instance of a processor which maps indications derived from the IMU data to care actions, where said care actions comprise at least one allocation of at least one organizational resource from among a set of available organizational resources which the organization allocates to its end-users or patients or members,
thereby to reliably and efficiently allocate available organizational resources to at least one organization's patients.

15. A computer program product, comprising a non-transitory tangible computer readable medium having computer readable program code embodied therein, said computer readable program code adapted to be executed to implement a computerized healthcare method providing healthcare remotely to end-users, the method comprising providing at least one instance of an interface uploading IMU data recorded by a healthcare providing organization O's end users' communication devices' accelerometers; and
providing at least one instance of a processor which maps indications derived from the IMU data to care actions, where said care actions comprise at least one allocation of at least one organizational resource from among a set of available organizational resources which the organization allocates to its end-users or patients or members,
thereby to reliably and efficiently allocate available organizational resources to at least one organization's patients.
